SEQUENCE LISTING (57 sequences)

SEQ ID NO: 1 - protein, 543 aa, synthetic construct
  source 1..543: note = Enhanced Beetle Luciferase (ELuc)
MEKREKNVVYGPEPKHPLGNFTAGEMLYNALHKHSHIPQAILDVMGNESLSYQEFFDTTVKLGQSLQNCGYKMNDVVSICAENNKRFFIPIISAWYIGMVVAPVNEDYIPDELCKVTGISKPILVFTTRKILPKVLEVKDRTNYIKRIIILDSEENLLGCESLHNFMSRYSDNNLQTFKPLHYDPVDQVAAILCSSGTTGLPKGVMQTHRNICVRLTHASDPRVGTQLIPGVSVLAYLPFFHAFGFSINLGYFMVGLRVVMLRRFNQEVFLKAIQDYEVRSVINVPSTILFLSKSPLVDKYDLSTLAELCCGAAPLAKEVAEIAVKRLNLPGIRCGYGLTESTSANIHTLHNEFKSGSLGKVTPYMAAKIIDRNTGEALGPNQVGELCIWGPMVTKGYVNNPQATKEAIDDDGWLHSGDFGYYDEDEYFYIVDRYKELIKYKGYQVAPVELEEILLQHPGIRDVAVVGIPDIEAGELPAGFVVKQPGAQLTAKEVYDFLAQRVSHSKYLRGGVRFVDSIPRNVTGKISRKELREALMEKASKL

SEQ ID NO: 2 - protein, 542 aa, synthetic construct
  source 1..542: note = Click Beetle Green Luciferase (CBG)
MVKREKNVIYGPEPLHPLEDLTAGEMLFRALRKHSHLPQALVDVVGDESLSYKEFFEATVLLAQSLHNCGYKMNDVVSICAENNTRFFIPVIAAWYIGMIVAPVNESYIPDELCKVMGISKPQIVFTTKNILNKVLEVQSRTNFIKRIIILDTVENIHGCESLPNFISRYSDGNIANFKPLHFDPVEQVAAILCSSGTTGLPKGVMQTHQNICVRLIHALDPRYGTQLIPGVTVLVYLPFFHAFGFHITLGYFMVGLRVIMFRRFDQEAFLKAIQDYEVRSVINVPSVILFLSKSPLVDKYDLSSLRELCCGAAPLAKEVAEVAAKRLNLPGIRCGFGLTESTSAIIQTLGDEFKSGSLGRVTPLMAAKIADRETGKALGPNQVGELCIKGPMVSKGYVNNVEATKEAIDDDGWLHSGDFGYYDEDEHFYVVDRYKELIKYKGSQVAPAELEEILLKNPCIRDVAVVGIPDLEAGELPSAFVVKQPGTEITAKEVYDYLAERVSHTKYLRGGVRFVDSIPRNVTGKITRKELLKQLLVKAGG

SEQ ID NO: 3 - protein, 543 aa, synthetic construct
  source 1..543: note = R. ohbai Luciferase (RoLuc)
MPNEIILHGAKPRDPLDLGTAGIQLYRALTNFSFLREALIDAHTEEVVSYADILENSCRLAKCYENYGLRQNSVISVCSENSTIFFYPVIAALYMGVITATVNDSYTERELLETLNISKPELVFCSKKAIKNMMALKRNVNFIKKVVLLDSKEDMGEAQCLSNFMARYSEPNLDVRNFKPRDFDAKEQVALIMSSSGTTGLPKGVVLTHRNLSVRFVHCKDPLFGTRTIPSTSILSIVPFHHAFGMFTTLSYFIVGLRVVLLKRFEEKFFLSTIEKYRIPTIVLAPPVMVFLAKSPLVDQYDLSSIREVATGGAPVGTEVAVAVAKRLKIGGILQGYGLTETCCAVLITPHDDVKTGSTGRVAPYVQAKIVDLTTGKSLGPNKRGELCFKSEIIMKGYFNNKQATEEAIDKEGWLHSGDVGYYDDDGHFFVVDRLKELIKYKGYQVAPAELEWLLLQHPSIKDAGVTGVPDEAAGELPGACIVLQEGKSLTEQEIIDYIAERVSPTKRIRGGVVFVDDIPKGATGKLVRSELRKLLAQKKSKL

SEQ ID NO: 4 - protein, 550 aa, synthetic construct
  source 1..550: note = Firefly Luciferase (FLuc)
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 5 - protein, 550 aa, synthetic construct
  source 1..550: note = Red Firefly Luciferase (RedF)
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRALCVRFSHARDPIFGNQIAPDTAILSVVPFHHGFGMFTTLGYLICGFRVVLMYRFEEELFLRSLQDYKIQTALLVPTLFSFLAKSTLIDKYDLSNLHEIASGGAPLSKEVGEAVAKRFHLPGIRQGYGLTETTSAILVTPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIRDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 6 - protein, 546 aa, synthetic construct
  source 1..546: note = P. hirtus Red Luciferase (RedLuc)
MEEENIVNGDRPRDLVFPGTAGLQLYQSLYKYSYITDGIIDAHTNEVISYAQIFETSCRLAVSLEKYGLDHNNVVAICSENNIHFFGPLIAALYQGIPMATSNDMYTEREMIGHLNISKPCLMFCSKKSLPFILKVQKHLDFLKKVIVIDSMYDINGVECVFSFVSRYTDHAFDPVKFNPKEFDPLERTALIMTSSGTTGLPKGVVISHRSITIRFVHSSDPIYGTRIAPDTSILAIAPFHHAFGLFTALAYFPVGLKIVMVKKFEGEFFLKTIQNYKIASIVVPPPIMVYLAKSPLVDEYNLSSLTEIACGGSPLGRDIADKVAKRLKVHGILQGYGLTETCSALILSPNDRELKKGAIGTPMPYVQVKVIDINTGKALGPREKGEICFKSQMLMKGYHNNPQATRDALDKDGWLHTGDLGYYDEDRFIYVVDRLKELIKYKGYQVAPAELENLLLQHPNISDAGVIGIPDEFAGQLPSACVVLEPGKTMTEKEVQDYIAELVTTTKHLRGGVVFIDSIPKGPTGKLMRNELRAIFAREQAKSKL

SEQ ID NO: 7 - protein, 171 aa, synthetic construct
  source 1..171: note = Nano Luciferase (NLuc)
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLCERILA

SEQ ID NO: 8 - protein, 311 aa, synthetic construct
  source 1..311: note = Renilla Luciferase (Renilla)
MTSKVYDPEQRKRMITGPQWWARCKQMNVLDSFINYYDSEKHAENAVIFLHGNAASSYLWRHVVPHIEPVARCIIPDLIGMGKSGKSGNGSYRLLDHYKYLTAWFELLNLPKKIIFVGHDWGACLAFHYSYEHQDKIKAIVHAESVVDVIESWDEWPDIEEDIALIKSEEGEKMVLENNFFVETMLPSKIMRKLEPEEFAAYLEPFKEKGEVRRPTLSWPREIPLVKGGKPDVVQIVRNYNAYLRASDDLPKMFIESDPGFFSNAIVEGAKKFPNTEFVKVKGLHFSQEDAPDEMGKYIKSFVERVLKNEQ

SEQ ID NO: 9 - protein, 219 aa, synthetic construct
  source 1..219: note = Metridia Luciferase (MetLuc)
MDIKVVFTLVFSALVQAKSTEFDPNIDIVGLEGKFGITNLETDLFTIWETMEVMIKADIADTDRASNFVATETDANRGKMPGKKLPLAVIMEMEANAFKAGCTRGCLICLSKIKCTAKMKVYIPGRCHDYGGDKKTGQAGIVGAIVDIPEISGFKEMAPMEQFIAQVDRCASCTTGCLKGLANVKCSELLKKWLPDRCASFADKIQKEVHNIKGMAGDR

SEQ ID NO: 10 - protein, 209 aa, synthetic construct
  source 1..209: note = Lucia Luciferase (Lucia)
MEIKVLFALICIAVAEAKPTEINEDLNIAAVASNFATTDLETDLFTNWETMNVISTDTEQVNTDADRGKLPGKKLPPDVLRELEANARRAGCTRGCLICLSHIKCTPKMKKFIPGRCHTYEGEKESAQGGIGEAIVDIPEIPGFKDKEPLDQFIAQVDLCADCTTGCLKGLANVQCSDLLKKWLPQRCTTFASKIQGRVDKIKGLAGDR

SEQ ID NO: 11 - protein, 185 aa, synthetic construct
  source 1..185: note = Gaussia Luciferase (GLuc)
MGVKVLFALICIAVAEAKPTENNEDFNIVAVASNFATTDLDADRGKLPGKKLPLEVLKEMEANARKAGCTRGCLICLSHIKCTPKMKKFIPGRCHTYEGDKESAQGGIGEAIVDIPEIPGFKDLEPMEQFIAQVDLCVDCTTGCLKGLANVQCSDLLKKWLPQRCATFASKIQGQVDKIKGAGGD

SEQ ID NO: 12 - protein, 311 aa, synthetic construct
  source 1..311: note = Green Renilla Luciferase (GrRenilla)
MASKVYDPEQRKRMITGPQWWARCKQMNVLDSFINYYDSEKHAENAVIFLHGNATSSYLWRHVVPHIEPVARCIIPDLIGMGKSGKSGNGSYRLLDHYKYLTAWFELLNLPKKIIFVGHDWGSALAFHYAYEHQDRIKAIVHMESVVDVIESWMGWPDIEEELALIKSEEGEKMVLENNFFVETLLPSKIMRKLEPEEFAAYLEPFKEKGEVRRPTLSWPREIPLVKGGKPDVVQIVRNYNAYLRASDDLPKLFIESDPGFFSNAIVEGAKKFPNTEFVKVKGLHFLQEDAPDEMGKYIKSFVERVLKNEQ

SEQ ID NO: 13 - protein, 2813 aa, synthetic construct
  source 1..2813: note = full VWF
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCQDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 14 - protein, 168 aa, synthetic construct
  source 1..168: note = A2 subunit
DVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLV

SEQ ID NO: 15 - protein, 304 aa, synthetic construct
  source 1..304: note = C-terminal Nluc-VWFA2-FLAG-His fusion protein
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLCERILAGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGDYKDDDDKLEHHHHHH

SEQ ID NO: 16 - protein, 304 aa, synthetic construct
  source 1..304: note = N-terminal Nluc-VWFA2-FLAG-His fusion protein
MDYKDDDDKGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLCERILALEHHHHHH

SEQ ID NO: 17 - protein, 310 aa, synthetic construct
  source 1..310: note = NlucC166F-VWFA2
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLFERILAGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGLPETGDYKDDDDKCLEHHHHHH

SEQ ID NO: 18 - protein, 315 aa, synthetic construct
  source 1..315: note = NlucC166F-VWFA2-Bigger Spacer-Cys
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLFERILAGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGSGGGSGGGSGGGSGGGSHHHHHHGGGC

SEQ ID NO: 19 - protein, 6 aa, synthetic construct
  source 1..6: note = six his
HHHHHH

SEQ ID NO: 20 - protein, 7 aa, synthetic construct
  source 1..7: note = seven his
HHHHHHH

SEQ ID NO: 21 - protein, 8 aa, synthetic construct
  source 1..8: note = eight his
HHHHHHHH

SEQ ID NO: 22 - protein, 8 aa, synthetic construct
  source 1..8: note = LE(H)6
LEHHHHHH

SEQ ID NO: 23 - protein, 9 aa, synthetic construct
  source 1..9: note = HA-tag
YPYDVPDYA

SEQ ID NO: 24 - protein, 8 aa, synthetic construct
  source 1..8: note = FLAG-tag
DYKDDDDK

SEQ ID NO: 25 - protein, 11 aa, synthetic construct
  source 1..11: note = Linker
GGGSGGGSGGG

SEQ ID NO: 26 - protein, 15 aa, synthetic construct
  source 1..15: note = Linker
GGGSGGGSGGGSGGG

SEQ ID NO: 27 - protein, 19 aa, synthetic construct
  source 1..19: note = Linker
GGGSGGGSGGGSGGGSGGG

SEQ ID NO: 28 - protein, 28 aa, synthetic construct
  source 1..28: note = Linker
GGGSGGGSGGGSGGGSGGGSGGGSGGGS

SEQ ID NO: 29 - protein, 16 aa, synthetic construct
  source 1..16: note = Linker
GGGSGGGSGGGLPETG

SEQ ID NO: 30 - protein, 5 aa, synthetic construct
  source 1..5: note = Linker
LPETG

SEQ ID NO: 31 - protein, 8 aa, synthetic construct
  source 1..8: note = Linker
GGGLPETG

SEQ ID NO: 32 - protein, 8 aa, synthetic construct
  source 1..8: note = Linker
LPETGGGG

SEQ ID NO: 33 - protein, 12 aa, synthetic construct
  source 1..12: note = Linker
GGGSLPETGGGG

SEQ ID NO: 34 - protein, 11 aa, synthetic construct
  source 1..11: note = extended recognition site
APNLVYMVTGN

SEQ ID NO: 35 - DNA, 30 bp, synthetic construct
  source 1..30: mol_type = other DNA; note = nucleic acid encoding C-Flag
gactacaaagacgatgacgacaagcttgaa

SEQ ID NO: 36 - DNA, 27 bp, synthetic construct
  source 1..27: mol_type = other DNA; note = nucleic acid encoding N-Flag
atggattacaaggatgacgacgataag

SEQ ID NO: 37 - DNA, 930 bp, synthetic construct
  source 1..930: mol_type = other DNA; note = nucleic acid encoding No 17
atggtgtttaccctggaagattttgtgggcgattgggaacaaacggctgcgtataatctggaccaggtactggaacagggcggggtatcgagtcttttacagaacctcgccgtttccgttacaccgatccaacgcatcgtccgtagcggggagaatgcattgaaaattgacattcacgttataattccgtacgaaggtctgagcgccgatcagatggcgcagatcgaagaagtttttaaagttgtgtatccggtagatgaccatcatttcaaagtcatcctgccatacggtacgctagtgattgatggcgtgacccctaacatgctcaattattttggccgcccctacgagggcattgcggtcttcgacggcaagaagattaccgtgaccggtacgctttggaacggcaacaaaattatcgatgaacgtttaattactccggatggatcaatgctgtttcgcgtgactattaatggagtgaccggttggcgtctgttcgagcgaatcttggcaggtggcggatccggtggcgggtcaggaggagggaatcgcaccaatacggggctggcattacgttacctgtccgatcatagttttttggttagccagggtgatcgtgagcaggctccgaacctggtgtatatggtgacaggcaacccagcctcggatgagatcaaacgtctccccggcgacattcaagtcgttccgatcggcgtagggcctaatgcgaacgtccaggaactggaacgcattggttggccgaacgcgccgattctaattcaagatttcgaaacccttccgcgcgaagcccctgacttagtgctgcagcggggtggcggatctggcggaggctctggtggcggactgccggaaactggtgattataaggatgacgacgataaatgcctcgagcaccaccaccaccaccac

SEQ ID NO: 38 - DNA, 945 bp, synthetic construct
  source 1..945: mol_type = other DNA; note = nucleic acid encoding No 18
atggtgtttaccctggaagattttgtgggcgattgggaacaaacggctgcgtataatctggaccaggtactggaacagggcggggtatcgagtcttttacagaacctcgccgtttccgttacaccgatccaacgcatcgtccgtagcggggagaatgcattgaaaattgacattcacgttataattccgtacgaaggtctgagcgccgatcagatggcgcagatcgaagaagtttttaaagttgtgtatccggtagatgaccatcatttcaaagtcatcctgccatacggtacgctagtgattgatggcgtgacccctaacatgctcaattattttggccgcccctacgagggcattgcggtcttcgacggcaagaagattaccgtgaccggtacgctttggaacggcaacaaaattatcgatgaacgtttaattactccggatggatcaatgctgtttcgcgtgactattaatggagtgaccggttggcgtctgttcgagcgaatcttggcaggtggcggatctggcggaggctctggtggcggaaatcgcaccaatacggggctggcattacgttacctgtccgatcatagttttttggttagccagggtgatcgtgagcaggctccgaacctggtgtatatggtgacaggcaacccagcctcggatgagatcaaacgtctccccggcgacattcaagtcgttccgatcggcgtagggcctaatgcgaacgtccaggaactggaacgcattggttggccgaacgcgccgattctaattcaagatttcgaaacccttccgcgcgaagcccctgacttagtgctgcagcggggtggtggttctggtggtggatctggtggcggtagtggtggtggtagtggtggtggaagtggggggggtagcggtggtggatcccaccaccaccaccaccacggtggcggttgt

SEQ ID NO: 39 - protein, 171 aa, synthetic construct
  source 1..171: note = NLuc C166F
MVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINGVTGWRLFERILA

SEQ ID NO: 40 - protein, 93 aa, synthetic construct
  source 1..93: note = Variation A2 domain #1
GNRTNTGLALRYLSDHSFLVSQGDREQAPNLVYMVTGNPASDEIKRLPGDIQVVPIGVGPNANVQELERIGWPNAPILIQDFETLPREAPDLV

SEQ ID NO: 41 - protein, 93 aa, synthetic construct
  source 1..93: note = Variation A2 domain #2
GNRTNTGLALRYLSDHSFLVSQGDREQAPNLVYMVTGYRLFKKISRLPGDIQVVPIGVGPNANVQELERIGWPNAPILIQDFETLPREAPDLV

SEQ ID NO: 42 - protein, 93 aa, synthetic construct
  source 1..93: note = Variation A2 domain #3
GNRTNTGLALRYLSDHSFLVSQGDREQAPNLVYMVTGYRLFEKISRLPGDIQVVPIGVGPNANVQELERIGWPNAPILIQDFETLPREAPDLV

SEQ ID NO: 43 - protein, 93 aa, synthetic construct
  source 1..93: note = Variation A2 domain #4
GNRTNTGLALRYLSDHSFLVSQGDREQAPNLVYMVTGYRLFEKILRLPGDIQVVPIGVGPNANVQELERIGWPNAPILIQDFETLPREAPDLV

SEQ ID NO: 44 - protein, 93 aa, synthetic construct
  source 1..93: note = Variation A2 domain #5
GNRTNTGLALRYLSDHSFLVSQGDREQAPNLVYMVTGWRLCERILRLPGDIQVVPIGVGPNANVQELERIGWPNAPILIQDFETLPREAPDLV

SEQ ID NO: 45 - protein, 93 aa, synthetic construct
  source 1..93: note = Variation A2 domain #6
GNRTNTGLALRYLSDHSFLVSQGDREQAPNLVYMVEGYRLFEKISRLPGDIQVVPIGVGPNANVQELERIGWPNAPILIQDFETLPREAPDLV

SEQ ID NO: 46 - protein, 93 aa, synthetic construct
  source 1..93: note = Variation A2 domain #7
GNRTNTGLALRYLSDHSFLVSQGDREQAPNLVYMVTGYRLFEEILRLPGDIQVVPIGVGPNANVQELERIGWPNAPILIQDFETLPREAPDLV

SEQ ID NO: 47 - protein, 93 aa, synthetic construct
  source 1..93: note = Variation A2 domain #8
GNRTNTGLALRYLSDHSFLVSQGDREQAPNLVYMVSGWRLFKKISRLPGDIQVVPIGVGPNANVQELERIGWPNAPILIQDFETLPREAPDLV

SEQ ID NO: 48 - protein, 10 aa, synthetic construct
  source 1..10: note = C-Flag
DYKDDDDKLE

SEQ ID NO: 49 - protein, 9 aa, synthetic construct
  source 1..9: note = N-Flag
MDYKDDDDK

SEQ ID NO: 50 - DNA, 915 bp, synthetic construct
  source 1..915: mol_type = other DNA; note = C-terminal Nluc-VWFA2-FLAG-His fusion protein
atggtgtttaccctggaagattttgtgggcgattgggaacagaccgcggcgtataacctggatcaggtgctggaacagggcggcgtgagcagcctgctgcagaacctggcggtgagcgtgaccccgattcagcgcattgtgcgcagcggcgaaaacgcgctgaaaattgatattcatgtgattattccgtatgaaggcctgagcgcggatcagatggcgcagattgaagaagtgtttaaagtggtgtatccggtggatgatcatcattttaaagtgattctgccgtatggcaccctggtgattgatggcgtgaccccgaacatgctgaactattttggccgcccgtatgaaggcattgcggtgtttgatggcaaaaaaattaccgtgaccggcaccctgtggaacggcaacaaaattattgatgaacgcctgattaccccggatggcagcatgctgtttcgcgtgaccattaacggcgtgaccggctggcgcctgtgcgaacgcattctggcgggaggaggatccggcggtgggtccggtggtggtaaccgcaccaacaccggcctggcgctgcgctatctgagcgatcatagctttctggtgagccagggcgatcgcgaacaggcgccgaacctggtgtatatggtgaccggcaatccggcgtctgatgaaattaaacgcctgccgggcgatattcaggtggtgccgattggcgtgggcccgaacgcgaacgtgcaggaactggaacgcattggctggccgaacgcgccgattctgattcaggattttgaaaccctgccgcgcgaagcgccggatctggtgctgcagcgcggtggcggatctggcggaggctctggtggcggagattataaagatgatgatgataaactcgagcaccaccaccaccaccactga

SEQ ID NO: 51 - DNA, 915 bp, synthetic construct
  source 1..915: mol_type = other DNA; note = N-terminal Nluc-VWFA2-FLAG-His fusion protein
atggattataaagatgatgatgataaaggtggcggatctggcggaggctctggtggcggaaaccgcaccaacaccggcctggcgctgcgctatctgagcgatcatagctttctggtgagccagggcgatcgcgaacaggcgccgaacctggtgtatatggtgaccggcaatccggcgtctgatgaaattaaacgcctgccgggcgatattcaggtggtgccgattggcgtgggcccgaacgcgaacgtgcaggaactggaacgcattggctggccgaacgcgccgattctgattcaggattttgaaaccctgccgcgcgaagcgccggatctggtgctgcagcgcggaggaggatccggcggtgggtccggtggtggtgtgtttaccctggaagattttgtgggcgattgggaacagaccgcggcgtataacctggatcaggtgctggaacagggcggcgtgagcagcctgctgcagaacctggcggtgagcgtgaccccgattcagcgcattgtgcgcagcggcgaaaacgcgctgaaaattgatattcatgtgattattccgtatgaaggcctgagcgcggatcagatggcgcagattgaagaagtgtttaaagtggtgtatccggtggatgatcatcattttaaagtgattctgccgtatggcaccctggtgattgatggcgtgaccccgaacatgctgaactattttggccgcccgtatgaaggcattgcggtgtttgatggcaaaaaaattaccgtgaccggcaccctgtggaacggcaacaaaattattgatgaacgcctgattaccccggatggcagcatgctgtttcgcgtgaccattaacggcgtgaccggctggcgcctgtgcgaacgcattctggcgctcgagcaccaccaccaccaccactga

SEQ ID NO: 52 - protein, 95 aa, synthetic construct
  source 1..95: note = Reduced A2 domain
NRTNTGLALRYLSDHSFLVSQGDREQAPNLVYMVTGNPASDEIKRLPGDIQVVPIGVGPNANVQELERIGWPNAPILIQDFETLPREAPDLVLQR

SEQ ID NO: 53 - protein, 550 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDLAYWDEDEHFFIVGRLKSLIKYKGYQVAPAELESILLQHPNIFDAGVAGLPDDDAGELPAAVVVLEHGKTMTEKEIVDYVASQVTTAKKLRGGVVFVDEVPKGLTGKRDARKIREILIKAKKGGKSKL

SEQ ID NO: 54 - protein, 689 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRALCVRFSHARDPIFGNQIAPDTAILSVVPFHHGFGMFTTLGYLICGFRVVLMYRFEEELFLRSLQDYKIQSALLVPTLFSFLAKSTLIDKYDLSNLHEIASGGAPLSKEVGEAVAKRFHLPGIRQGYGLTETTSAILITPKGDDKPGAVGKVVPFFEAKVVDLDTGKTLGVNQRGELCVRGPMIMSGYVNNPEATNALIDKDGWLHSGDLAYWDEDEHFFIVGRLKSLIKYKGYQVAPAELESILLQHPNIFDAGVAGLPDDDAGELPAAVVVLEHGKTMTEKEIVDYVASQVTTAKKLRGGVVFVDEVPKGLTGKRDARKIREILIKAKKGGKSKLGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGLPETGDYKDDDDKCLEHHHHHH

SEQ ID NO: 55 - protein, 544 aa, synthetic construct
MADKNILYGPEPFYPLEDGTAGEQMFDALSRYADIPGCIALTNAHTKENVLYEEFLKLSCRLAESFKKYGLKQNDTIAVCSENGLQFFLPVIASLYLGIIVAPVNDKYIERELIHSLGIVKPRIVFCSKNTFQKVLNVKSKLKSIETIIILDLNEDLGGYQCLNNFISQNSDSNLDVKKFKPYSFNRDDQVALIMFSSGTTGLPKGVMLTHKNIVARFSLAKDPTFGNAINPTTAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPKGDAKPGSTGKIVPFHAVKVVDPTTGKILGPNEPGELYFKGPMIMKGYYNNEEATKAIIDNDGWLRSGDIAYYDNDGHFYIVDRLKSLIKYKGYQVAPAEIEGILLQHPYIVDAGVTGIPDEAAGELPAAGVVVQTGKYLNEQIVQDYVASQVSTAKWLRGGVKFLDEIPKGSTGKIDRKVLRQMFEKHTNG

SEQ ID NO: 56 - protein, 683 aa, synthetic construct
MADKNILYGPEPFYPLEDGTAGEQMFDALSRYADIPGCIALTNAHTKENVLYEEFLKLSCRLAESFKKYGLKQNDTIAVCSENGLQFFLPVIASLYLGIIVAPVNDKYIERELIHSLGIVKPRIVFCSKNTFQKVLNVKSKLKSIETIIILDLNEDLGGYQCLNNFISQNSDSNLDVKKFKPYSFNRDDQVALIMFSSGTTGLPKGVMLTHKNIVARFSLAKDPTFGNAINPTTAILTVIPFHHGFGMMTTLGYFTCGFRVVLMHTFEEKLFLQSLQDYKVESTLLVPTLMAFLAKSALVEKYDLSHLKEIASGGAPLSKEIGEMVKKRFKLNFVRQGYGLTETTSAVLITPKGDAKPGSTGKIVPFHAVKVVDPTTGKILGPNEPGELYFKGPMIMKGYYNNEEATKAIIDNDGWLRSGDIAYYDNDGHFYIVDRLKSLIKYKGYQVAPAEIEGILLQHPYIVDAGVTGIPDEAAGELPAAGVVVQTGKYLNEQIVQDYVASQVSTAKWLRGGVKFLDEIPKGSTGKIDRKVLRQMFEKHTNGGGGSGGGSGGGNRTNTGLALRYLSDHSFLVSQGDREQAPNLVYMVTGNPASDEIKRLPGDIQVVPIGVGPNANVQELERIGWPNAPILIQDFETLPREAPDLVLQRGGGSGGGSGGGLPETGDYKDDDDKCLEHHHHHH

SEQ ID NO: 57 - protein, 13 aa, synthetic construct
GGGSGGGSGGGTS

(19) United States
(12) Patent Application Publication
(10) Pub. No.: US 2026/0218270 A1
(43) Pub. Date: Jul. 30, 2026

(54) Luminescent detection of ADAMTS13

(71) Applicant: Enzyre B.V., Nijmegen (NL)

(72) Inventors: Alida Lidwina Maria Bloom, Nijmegen (NL); Mark van Geffen, Nijmegen (NL); Waander Laurens van Heerde, Nijmegen (NL); Zixuan Li, Nijmegen (NL); Jona Merx, Nijmegen (NL); Theodora Johannes Steeghs, Nijmegen (NL); Cornelis van 't Veer, Nijmegen (NL); Thomas Jan Boltje, Nijmegen (NL); Floris Petrus Johannes Theodorus Rutjes, Nijmegen (NL); Daan Sondag, Nijmegen (NL)

(73) Assignee: Enzyre B.V., Nijmegen (NL)

(21) Appl. No.: 19/148,325

(22) PCT Filed: Jan. 18, 2024
(86) PCT No.: PCT/EP2024/051172

§ 371 Date: Jul. 16, 2025

(30) Foreign Application Priority Data
Jan. 18, 2023 (EP) 23152306.9

(51) Int. Cl. C12Q 1/66; C07K 14/755; C12N 9/02; C12Q 1/37

(52) U.S. Cl. CPC C12Q 1/66; C07K 14/755; C12N 9/0069; C12Q 1/37; C12Y 113/12013; C07K 2319/21; C07K 2319/61

(57) ABSTRACT

The present invention relates to substrate molecules suitable for monitoring the enzyme ADAMTS13 via chemiluminescence. The substrates are particularly useful for assaying this enzyme in a sample such as a blood sample.

### FIELD OF THE INVENTION

The present invention relates to substrate molecules suitable for monitoring the enzyme ADAMTS13 via chemiluminescence. The substrates are particularly useful for assaying this enzyme in a sample such as a blood sample.

### BACKGROUND ART

Coagulation of blood, also known as clotting, is the transformation of blood from a liquid to a gel, resulting in a blood clot. Coagulation is part of the hemostasis process and ultimately prevents excessive blood loss. Coagulation begins very soon after endothelium lining of a vessel is compromised. Exposure of the subendothelial space leads to the binding of plasma Factor VII (FVII) to tissue factor, which ultimately leads to fibrin formation. In so-called primary hemostasis, platelets immediately form a plug at the site of injury. So-called secondary hemostasis occurs simultaneously: coagulation factors respond in a complex cascade to form fibrin strands, which strengthens the platelet plug. Coagulation is highly conserved throughout biology.

ADAMTS13 [the 13th member of A Disintegrin And Metalloprotease with ThromboSpondin type 1 motif] is a zinc metalloprotease which limits platelet aggregation and microthrombi formation in the microcirculation by cleaving Von Willebrand Factor [VWF] between Tyrosine 1605-Methionine 1606 [Tyr1605-Met1606] to generate a series of small molecular weight multimers. A deficiency of ADAMTS13 either because of an inherited mutation within the ADAMTS13 gene or the development of an autoantibody leads to the potentially lethal syndrome of Thrombotic Thrombocytopenic Purpura [TTP]. ADAMTS13 is relevant in two disease area's: TTP and in the case of vaso-occlusive crises (VOC) related to sickle cell disease (SCD). For the latter disease area it is especially important that point-of-care devices are developed since SCD patients mostly are found in sub-Saharan Africa, living in remote areas and thereby benefitting from a point of care device that easily can detect their ADAMTS13 level upon treatment for their VOC.

Methods for quantifying blood clotting factors are well known (see S. S. van Berkel, PhD thesis chapter 1, 2008, Radboud University Nijmegen). Methods for global hemostasis assays, a newer class of assays, are described by M. van Geffen (PhD Thesis chapters 2 and 3, 2012, Radboud University Nijmegen). Traditionally, in these known methods for quantification of blood clotting factors, fibrin formation is measured as a read out. Alternatively, later-developed chromogenic probes based on paranitroaniline conjugated to a peptide release free paranitroaniline (pNA) when the peptide is cleaved by an enzyme such as thrombin or FXa. The resulting color can be quantified and is a measure of enzyme activity. Examples are the commercially available S-2765 from Chromogenix, and the commercially available S-2222 from Chromogenix.

Chromogenic tests have a disadvantage: because their methods depend upon the measurement of optical density, they cannot be carried out in a mixture that would become turbid due to clot formation, and the chromogenic yellow color interferes with the intrinsic yellow color of plasma. Therefore they should be carried out in defibrinated and consequently platelet poor plasma. Also they require subsampling because they cannot be measured in a continuous setting. Going from platelet poor plasma (PPP) to platelet rich plasma (PRP) to whole blood, the physiological system becomes more representative of what happens in the body though concomitantly, technically more difficult to assess.

Application of fluorogenic substrates made measurement in non defibrinated, platelet rich plasma and whole blood possible, and thus brought the assay system one step nearer to the physiological system while allowing continuous monitoring. The Van Berkel thesis cited above discusses the development of fluorogenic probes for thrombin.

The fluorometric probe ab204711 is commercially available (from Abcam PLC, UK). This Factor Xa Activity Assay Kit (Fluorometric) (ab204711) utilizes the ability of Factor Xa to cleave a synthetic substrate thereby releasing a fluorophore which can be quantified by fluorescence readers. A similar kit is available from Merck (catalogue number MAK238-1 KT).

Other commercially available substrates are the SensoLyte® Rh110 Factor Xa Assay Kit from Eurogentec, which also uses a fluorogenic substrate that generates a fluorophore that can be detected after FXa cleavage of the substrate. The SensoLyte® 520 Factor Xa Assay Kit uses a 5-FAM/QXL™ 520 fluorescence resonance energy transfer (FRET) peptide, wherein fluorescence of 5-FAM is quenched by QXL™ 520. When FXa cleaves the intact peptide into two separate fragments, fluorescence of 5-FAM is recovered. This FRET peptide shows less interference from autofluorescence of test compounds and cellular components. WO2006/072602 describes the use of multiple fluorogenic substrates with different characteristics to allow the detection of several products in one sample.

Fluorogenic substrates have disadvantages: commercial platforms for analysis of the coagulation system generally do not support fluorometric analysis, thus requiring additional instrumentation. In addition, there is a desire to implement all coagulation tests wherever possible on one analyzer to simplify testing and minimize labor. The use of a separate instrument for measuring global assays thus reduces its applicability as a routine method. Fluorescent signals also have the drawback of not being linear with product concentration due to inner-filter effects and quenching effects.

Luminescent substrates do not have these disadvantages. They are more sensitive than chromogenic or fluorogenic substrates and do not require complex filters or excitation sources. U.S. Pat. No. 5,035,999 relates to luminescent substrates, but these substrates are not suitable for measurement in watery solutions (such as plasma), and neither for continuous measurement. WO2012096566 relates to substrates for thrombin or plasmin. Cosby et al. (“Custom enzyme substrates for luciferase-based assays”, Cell Notes, Issue 18 pages 9-11, 2007) relates to luminescent substrates, but these are not suitable for measurement in watery solutions (such as plasma) and neither for continuous measurement. Poor solubility often requires organic co-solvents that detract from the physiological conditions of an assay, or it requires larger volumes of sample or the addition of larger volumes of reagents.

It would be desirable for many clinical cases (e.g. in pediatric blood withdrawal, point of care monitoring in the home situation, in outbound critical care situations using an ambulance or helicopter) to have diagnostic tests for clotting factors that require a smaller reaction volume, thus requiring less blood. The system should be rather simple in its design and not require sophisticated technology, making it applicable in a (disposable) all in one point of care device. Moreover, a probe should allow a wide dynamic range, preferably over the three orders of magnitude offered by existing assays. The probe should be specific for its enzyme, such as ADAMTS13, and sensitive to allow its use in small sample volumes. The enabling of real-time measurement would improve flexibility of the assays in which these new probes could be used.

A number of different methods and approaches have evolved to measure ADAMTS13 activity and to detect the present of any inhibitory antibodies. Several of these are based on antigen detection, on gel electrophoresis, on western blotting, on chromogenic probes, or on fluorescence with or without FRET. These assays all have downsides, such as overestimations of ADAMTS13 levels, or being laborious and time-consuming (J. Evan Sadler, Blood. 2008 Jul. 1; 112(1): 11-18. DOI: 10.1182/blood-2008-02-078170). Without access to an improved ADAMTS13 assay, clinician must make a diagnosis and initiate therapy of for instance TTP without complete information.

A need exists for a new assay for measuring ADAMTS13 generation and/or measurement of other blood clotting factors or their activity, which does not have the above indicated drawbacks, that is it should be simpler and it should be able to measure the generation of blood clotting and fibrinolytic factors in a direct manner, preferably in a linear mode. It is an object of the present invention to provide substrates and methods for such an assay.

### SUMMARY OF THE INVENTION

The inventors found that the tyrosine methionine motif of Von Willebrand factor (VWF) can be used to indirectly release a polypeptide that has luciferase activity. The released polypeptide can generate a luminescent signal when contacted with a suitable substrate. Accordingly, the invention provides a polypeptide comprising a first sequence and a second sequence, wherein the first sequence encodes a polypeptide that has luciferase activity, and wherein the second sequence encodes a von Willebrand factor (VWF) domain that comprises a tyrosine-methionine recognition site for ADAMTS13. Preferably the first sequence encodes a polypeptide that has the activity of Enhanced Beetle Luciferase (ELuc), Click Beetle Green Luciferase (CBG), R. ohbai Luciferase (RoLuc), Firefly Luciferase (FLuc), Red Firefly Luciferase (RedF), P. hirtus Red Luciferase (RedLuc), Nano Luciferase (NLuc), Renilla Luciferase (Renilla), Metridia Luciferase (MetLuc), Lucia Luciferase (Lucia) Gaussia Luciferase (GLuc), or Green Renilla Luciferase (GrRenilla), more preferably it has the activity of NLuc. The first sequence preferably has at least 70% sequence identity with any one of SEQ ID NOs: 1-12, preferably with SEQ ID NO: 7, preferably at least 90% sequence identity, more preferably at least 98% sequence identity. In some embodiments the first sequence does not comprise a cysteine residue, preferably it has at least 70% sequence identity with SEQ ID NO: 39, preferably at least 90% sequence identity, more preferably at least 98% sequence identity.

In preferred embodiments, the second sequence has at least 70% sequence identity with any one of SEQ ID NOs: 13-14, preferably with SEQ ID NO: 14, preferably at least 90% sequence identity, more preferably at least 98% sequence identity. The second sequence preferably encodes the A2 domain of VWF, wherein the tyrosine-methionine recognition site for ADAMTS13 comprises a tyrosine at position 105, 106, 107, 108, 109, 110, or 111, wherein that tyrosine is directly followed by a methionine, optionally wherein the tyrosine-methionine recognition site is directly flanked by two valine residues.

In preferred embodiments the first sequence is N-terminal to the second sequence, or the second sequence is N-terminal to the first sequence, more preferably wherein the second sequence is N-terminal to the first sequence.

In preferred embodiments the polypeptide comprises a third sequence that encodes a first recognition tag, and optionally comprises a fourth sequence that encodes a second recognition tag, wherein the second recognition tag is distinct from the first recognition tag. Preferably the recognition tag is an epitope tag, an affinity-tag, a cysteine residue, or an aptamer-tag, more preferably the recognition tag is a histidine-tag or a FLAG-tag or a cysteine residue. Preferably the first sequence and the second sequence are separated by a linker sequence, wherein the linker sequence comprises 1-40 amino acids, preferably 3-30 amino acids.

In preferred embodiments the polypeptide comprises 200-500 amino acids, preferably 250-350 amino acids, more preferably 265-325 amino acids, optionally wherein the first sequence has a length of 150-450 amino acids, optionally wherein the second sequence has a length of 50-200 amino acids, optionally wherein a third sequence that encodes a first recognition tag is present and has a length of 4-25 amino acids, optionally wherein the fourth sequence that encodes a second recognition tag is present and has a length of 4-25 amino acids, optionally wherein one or more linkers are present and each linker sequence has a length of 1-30 amino acids.

In a preferred embodiment the polypeptide comprises a third sequence that encodes a first recognition tag, and a fourth sequence that encodes a second recognition tag, wherein the second recognition tag is distinct from the first recognition tag, and preferably wherein the order of the sequences within the polypeptide, from N-terminus to C-terminus, is either
i) the third sequence, the second sequence, the first sequence, and the fourth sequence; or
ii) the first sequence, the second sequence, the third sequence, and the fourth sequence.

In some embodiments the polypeptide has at least 95% sequence identity to SEQ ID NOs: 15-18, preferably to SEQ ID NO: 15, 17, or 18, preferably at least 98% sequence identity. In some embodiments the polypeptide has at least 95% sequence identity to SEQ ID NOs: 15-18 or 54 or 56, preferably to SEQ ID NO: 15, 17, 18, 54, or 56, preferably at least 98% sequence identity. In preferred embodiments the polypeptide comprises a third sequence that encodes a first recognition tag, wherein the first recognition tag is a cysteine residue. Preferably the polypeptide comprises a fourth sequence that encodes a second recognition tag, wherein the second recognition tag is distinct from the first recognition tag. Preferably the polypeptide comprises
a third sequence that encodes a first recognition tag, and
a fourth sequence that encodes a second recognition tag, wherein the second recognition tag is distinct from the first recognition tag, and an optional fifth sequence that encodes a third recognition tag, wherein the third recognition tag is distinct from the first and second recognition tags, preferably
wherein the order of the sequences within the polypeptide, from N-terminus to C-terminus, is either
i) the first sequence, the second sequence, the fourth sequence, and the third sequence; or
ii) the first sequence, the second sequence, the fifth sequence, the third sequence, and the fourth sequence.

In particularly preferred embodiments of the polypeptide, the first sequence has the activity of NLuc, the second sequence encodes the A2 domain of VWF, the third sequence is a cysteine residue, the fourth sequence is a histidine-tag, and, the fifth sequence is a FLAG-tag.

Also provided is a nucleic acid construct comprising a sequence that encodes a polypeptide as defined above. Also provided is a device for measuring luminescence, the device comprising a polypeptide as defined above, preferably wherein the device is a point of care device.

Also provided is a method for quantifying ADAMTS13 in a sample, the method comprising the steps of:
a) contacting the sample with a composition comprising a polypeptide as defined above to release a polypeptide fragment with luciferase activity; wherein the polypeptide is preferably freely dissolved;
b) separating the polypeptide fragment with luciferase activity from the remainder of the polypeptide as defined above;
c) contacting the fragment with luciferase activity with a suitable substrate; and
d) determining the relative light intensity generated by the fragment with luciferase activity.

More preferably the method comprises the steps of:
a) contacting the sample with a composition comprising a polypeptide as defined above, wherein the polypeptide is immobilized in an assay container, to release a polypeptide fragment with luciferase activity;
b) separating the polypeptide fragment with luciferase activity from the remainder of the polypeptide as defined above;
c) contacting the fragment with luciferase activity with a suitable substrate and optionally contacting the immobilized polypeptide in the assay container with a suitable substrate; and
d) determining the relative light intensity generated by the fragment with luciferase activity and optionally determining the relative light intensity generated by the immobilized polypeptide in the assay container.

### DESCRIPTION OF EMBODIMENTS

Provided is a polypeptide comprising a first sequence and a second sequence, wherein the first sequence encodes a polypeptide that has luciferase activity, and wherein the second sequence encodes a von Willebrand factor (VWF) domain that comprises a tyrosine-methionine recognition site for ADAMTS13. This is referred to herein as a polypeptide according to the invention. The presence of the tyrosine-methionine recognition site for ADAMTS13 allows the separation of the polypeptide into two fragments, one of which is a polypeptide fragment with luciferase activity. This is the fragment comprising the first sequence. It was found that the activity of this fragment is greatly enhanced, or even ‘switched on’ at all, only after the recognition site for ADAMTS13 is cleaved. This allows the chemiluminescent detection of ADAMTS13 activity.

First Sequence—Polypeptide that has Luciferase Activity

Luciferase is a generic term for the class of oxidative enzymes that produce bioluminescence using luciferin as a substrate. Luciferases do not require an external light source, but do require luciferin and 02, and often also ATP. Mg²⁺ is known to increase luminescent yield of luciferases. Luciferases and their assays are known in the art, as are suitable conditions for their activity. The polypeptide should be capable of converting an aminoluciferin or a related substrate into its oxidated analogue, under emission of a light quant.

The first sequence preferably encodes a polypeptide that has the activity of Enhanced Beetle Luciferase (ELuc), Click Beetle Green Luciferase (CBG), R. ohbai Luciferase (RoLuc), Firefly Luciferase (FLuc), Red Firefly Luciferase (RedF), P. hirtus Red Luciferase (RedLuc), Nano Luciferase (NLuc), Renilla Luciferase (Renilla), Metridia Luciferase (MetLuc), Lucia Luciferase (Lucia) Gaussia Luciferase (GLuc), or Green Renilla Luciferase (GrRenilla), most preferably it has the activity of NLuc. Accordingly, the first sequence preferably has at least 70% sequence identity with any one of SEQ ID NOs: 1-12 (which represent the above luciferase enzymes, respectively), more preferably with SEQ ID NO: 7 (NLuc), more preferably at least 90% sequence identity, even more preferably at least 98% sequence identity. SEQ ID NO: 7 is most preferred from amongst SEQ ID NOs: 1-12. In some embodiments the first sequence has 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity with any one of SEQ ID NOs: 1-12. In particular embodiments the first sequence has 100% sequence identity with any one of SEQ ID NOs: 1-12 with the proviso that optionally 1, 2, or 3 amino acids are substituted by other amino acids. Preferably 1 or 2 amino acids are substituted, more preferably 1 amino acid is substituted. Preferred examples of firefly luciferase are optimised P. photuris firefly luciferase also known as YY5 (SEQ ID NO: 53) and optimised P. pennsylvanica firefly luciferase also known as Luc90 (SEQ ID NO: 55). In highly preferred embodiments the luciferase is firefly luciferase or Nano Luciferase, more preferably it is represented by one of SEQ ID NOs: 53, 55, or 7, or has at least 70%, preferably 80%, more preferably 90%, more preferably 95%, most preferably 98% or 100% sequence identity therewith.

In preferred embodiments the first sequence does not comprise a cysteine residue, more preferably wherein it has at least 70% sequence identity with SEQ ID NO: 39, still more preferably at least 90% sequence identity, still more preferably at least 98% sequence identity, most preferably it has 100% sequence identity. Preferably all cysteine residues, if any, are substituted by a residue selected from glycine, alanine, valine, and phenylalanine, more preferably phenylalanine. Accordingly a preferred version of SEQ ID NO: 7 is C166F (SEQ ID NO: 39).

Many luciferases are known in the art. They can be commercially obtained from manufacturers such as Promega, Sigma, and the like. The polypeptide that has luciferase activity may be a native, a recombinant, or a mutant luciferase. Said mutant luciferase may be a modified luciferase comprising one or more amino acid substitutions, amino acid deletions, or amino acid insertions, as long as it retains its luciferase activity. Preferably the polypeptide that has luciferase activity has at least 25%, 50%, 75% of the luciferase activity of the native (recombinant) luciferase. In some embodiments the activity is at least 80%, 85%, 90%, or 95% of the activity of the polypeptide encoded by SEQ ID NO: 7.

The first sequence preferably has a length of 100-500 amino acids. More preferably the first sequence has a length of 150-450 amino acids, still more preferably of 150-250 amino acids, more preferably of 150-200 amino acids, even more preferably of 160-190, more preferably 170-180 amino acids, such as 171 amino acids.

For ease of manufacture, synthesis, or handling, it can be convenient to connect the polypeptide that has luciferase activity to a linker sequence. Linker sequences can consist of a mere single amino acid such as a single glycine residue, and they can also be longer sequences. The function of a linker sequence is to separate relevant domains from one another, and linkers are therefore often relatively simple in their design. A skilled person can recognize a linker sequence.

In preferred embodiments, the linker comprises only glycine, alanine, serine, and/or threonine residues, more preferably only glycine, alanine, and/or serine residues, most preferably only glycine and/or serine residues. Preferably a linker comprises predominantly glycine residues, such as comprising at least 50% glycine residues. For reasons related to solvent interactions, serine or threonine residues are attractive. Preferred linkers comprise at least one serine or threonine residue. More preferably, a linker comprises at least 10%, more preferably at least 25% serine residues. When a linker has a length of at least 4 amino acids, the linker preferably has at least 1 serine or threonine residue. Suitable linkers are represented by GGG and by SEQ ID NOs: 25-33 and optionally 57, preferably GGG or SEQ ID NO: 25.

In preferred embodiments a linker sequence comprises 1-40 amino acids, preferably 3-30 amino acids. Preferably the linker comprises 3-25, 4-22, 5-19, 6-18, 7-15, 9-13, or 10-11 amino acids. A linker can suitably separate the first sequence and the second sequence. When present, a linker preferably also separates the third sequence and the remainder of the polypeptide according to the invention. When present, a linker preferably also separates the fourth sequence and the remainder of the polypeptide according to the invention. When present, a linker preferably also separates the fifth sequence and the remainder of the polypeptide according to the invention.

In preferred embodiments the first sequence and the second sequence are separated by a linker sequence, wherein the linker sequence comprises 1-40 amino acids, preferably 3-30 amino acids.

### Second Sequence—Von Willebrand Factor (VWF) Domain

The second sequence encodes a VWF domain, and it can thus serve as a substrate for ADAMTS13. It was found that the connection between the first domain and the second domain hindered the enzymatic activity of the first domain, and thus the polypeptide according to the invention allowed the detection of ADAMTS13 activity by correlating it with luciferase activity.

Von Willebrand factor (VWF, represented by SEQ ID NO: 13) is a blood glycoprotein involved in hemostasis, specifically, platelet adhesion. It is a large multimeric glycoprotein that is present in blood plasma and produced constitutively as ultra-large VWF in endothelium (in the Weibel-Palade bodies), megakaryocytes (α-granules of platelets), and subendothelial connective tissue. The basic VWF monomer is a 2050-amino acid protein. Monomers contain a number of specific domains with a specific function, for instance the D′/D3 domain, which binds to factor VIII (von Willebrand factor type D domain); the A1 domain, which binds to platelet GPIb-receptor, heparin, and possibly collagen; the A2 domain, which must partially unfold to expose the buried cleavage site for the specific ADAMTS13 protease that inactivates VWF by making smaller multimers; the A3 domain, which binds to collagen (von Willebrand factor type A domain); the C4 domain, in which the RGD motif binds to platelet integrin αIIbβ3 when this is activated (von Willebrand factor type C domain); the other C domains, which may interact in ER dimers: the larger protein show six beads of (C and C-like) domains under cryo-EM; the “cystine knot” domain (at the C-terminal end of the protein), which VWF shares with platelet-derived growth factor (PDGF), transforming growth factor-β (TGFβ) and β-human chorionic gonadotropin (PHCG). Not all features of VWF are required for polypeptides according to the invention. It was found that the A2 domain (represented by SEQ ID NO: 14) is most relevant.

The second sequence comprises a tyrosine-methionine recognition site for ADAMTS13. The tyrosine and methionine are directly adjacent. The tyrosine is N-terminal to the methionine (it is YM). This recognition site is cleaved by hydrolysis between the tyrosine and the methionine residues. Preferably the recognition site is directly flanked by valine residues. More preferably the recognition site has at least 7 out of 11, more preferably at least 8 out of 11, still more preferably at least 9 out of 11, still more preferably at least 10 out of 11, most preferably all 11 residues in common with SEQ ID NO: 34, with the proviso that at least the tyrosine-methionine recognition site is present.

Preferably the second sequence has at least 70% sequence identity with any one of SEQ ID NOs: 13-14 or 40-47 or 52, more preferably with SEQ ID NO: 14 or 52, most preferably with SEQ ID NO: 52, preferably at least 90% sequence identity, more preferably at least 98% sequence identity. In some embodiments the second sequence has 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity with any one of SEQ ID NOs: 13-14 or 40-47 or 52. SEQ ID NO: 52 is most preferred. The sequence identity is preferably over the entire length of SEQ ID NO: 52.

In preferred embodiments the second sequence encodes the A2 domain of VWF, wherein the tyrosine-methionine recognition site for ADAMTS13 comprises a tyrosine at position 105, 106, 107, 108, 109, 110, or 111, wherein that tyrosine is directly followed by a methionine, optionally wherein the tyrosine-methionine recognition site is directly flanked by two valine residues. Herein, the positions are preferably as found in SEQ ID NO: 14. Preferably the tyrosine is at position 106, 107, 108, 109, or 110, more preferably 107, 108, or 109, most preferably 108. Position 108 of SEQ ID NO:14 corresponds to position 32 of SEQ ID NO: 52.

The second sequence preferably has a length of 50-250 amino acids. More preferably the second sequence has a length of 60-200 amino acids, still more preferably of 70-150 amino acids, more preferably of 80-115 amino acids, even more preferably of 85-110, more preferably 90-100 amino acids, such as 94 amino acids. The inventors found that a shorter sequence such as the one represented by SEQ ID NO: 52 is also an adequate substrate for ADAMTS13. Accordingly the invention provides a polypeptide comprising a sequence that has at least 90% sequence identity, more preferably at least 98% sequence identity with SEQ ID NO: 52, wherein the polypeptide comprises at most 150, preferably at most 130, more preferably at most 105, most preferably at most 100 contiguous amino acids of the A2 domain of VWF (preferably such as represented by SEQ ID NO: 14).

### Further Sequences—Recognition Tags

Polypeptides can be made more versatile by introduction of recognition tags. Such tags are widely known, and can aid in purification (for instance a histidine-tag) or recognition (for instance a FLAG-tag) or further reactivity (for instance a cysteine residue). In this context a tag can be as small as a single residue, for instance in the case of the cysteine residue described above. In general, tags are designed to be small so as to not influence the overall behaviour of the polypeptide.

In preferred embodiments the polypeptide comprises a third sequence that encodes a first recognition tag, and optionally comprises a fourth sequence that encodes a second recognition tag, and optionally comprises a fifth sequence that encodes a third recognition tag, wherein the second recognition tag is distinct from the first recognition tag, and wherein the second recognition tag is distinct from the third recognition tag, and wherein the first recognition tag is distinct from the third recognition tag. It is generally not useful to feature multiple instances of the same tag. In some embodiments, the polypeptide comprises a third sequence that encodes a first recognition tag, and comprises a fourth sequence that encodes a second recognition tag. In some embodiments, the polypeptide comprises a third sequence that encodes a first recognition tag, and comprises a fourth sequence that encodes a second recognition tag, and comprises a fifth sequence that encodes a third recognition tag.

Suitable recognition tags are an epitope tag, an affinity-tag, a cysteine residue, or an aptamer-tag, preferably the recognition tag is a histidine-tag or a FLAG-tag or a cysteine residue.

An epitope tag is a tag that can be recognized by an antibody. An example of an epitope tag is a FLAG-tag, which is an artificial antigen to which specific, high affinity monoclonal antibodies have been developed. This makes epitope tags (such as FLAG-tag) suitable for purification of the linked polypeptide (for instance via affinity chromatography) or for immobilization thereof (on a surface coated with FLAG-binding antibodies). Suitable FLAG-tags are SEQ ID NOs: 24, 48, and 49, preferably 24.

An affinity-tag is a tag that can bind a material. Affinity-tags allow polypeptides to be purified from for instance their crude biological source using an affinity technique such as by using histidine-tag, chitin binding protein (CBP), maltose binding protein (MBP), Strep-tag, or glutathione-S-transferase (GST). Preferred affinity-tags are histidine-tags. Such tags are widely known. Suitable histidine tags are represented by SEQ ID NOs: 19-22.

When a recognition tag is a cysteine residue, it is preferred that the polypeptide does not comprise other free cysteine residues, i.e. cysteine residues that are not comprised in a disulfide bridge. A skilled person knows how to assess this, for instance by using bioinformatics or information from crystal structures. Any free cysteine residues are preferably substituted by other amino acids, such as described elsewhere herein.

In preferred embodiment is provided the polypeptide according to the invention, wherein the polypeptide comprises a third sequence that encodes a first recognition tag, wherein the first recognition tag is a cysteine residue. It is even more preferred if this polypeptide comprises a fourth sequence that encodes a second recognition tag, wherein the second recognition tag is distinct from the first recognition tag. This second recognition tag is preferably a histidine-tag. Most preferably this polypeptide also comprises a fifth sequence that encodes a third recognition tag, and this third recognition tag is preferably a FLAG-tag.

### Architectures of the Polypeptide

In some embodiments the first sequence is N-terminal to the second sequence. In some embodiments the second sequence is N-terminal to the first sequence. When the polypeptide is intended for antibody immobilization using antibodies against the second domain, preferably the second sequence is N-terminal to the first sequence. When the polypeptide is intended for immobilization using a recognition tag, preferably the first sequence is N-terminal to the second sequence.

In preferred embodiments the polypeptide comprises 200-500 amino acids, preferably 250-350 amino acids, more preferably 265-325 amino acids, optionally wherein the first sequence has a length of 150-450 amino acids, optionally wherein the second sequence has a length of 50-200 amino acids, optionally wherein a third sequence that encodes a first recognition tag is present and has a length of 1-25, preferably 4-25 amino acids, optionally wherein the fourth sequence that encodes a second recognition tag is present and has a length of 4-25 amino acids, optionally wherein one or more linkers are present and each linker sequence has a length of 1-30 amino acids.

In preferred embodiments the polypeptide comprises 250-350 amino acids, more preferably 265-325 amino acids, wherein the first sequence has a length of 150-450 amino acids, wherein the second sequence has a length of 50-200 amino acids, wherein a third sequence that encodes a first recognition tag is present and has a length of 1-25 amino acids, wherein the fourth sequence that encodes a second recognition tag is present and has a length of 4-25 amino acids, optionally wherein one or more linkers are present and each linker sequence has a length of 1-30 amino acids.

In preferred embodiments the polypeptide comprises 265-325 preferably 302-305 most preferably 304 amino acids, wherein the first sequence has a length of 165-180 preferably 169-173 more preferably 171 amino acids, wherein the second sequence has a length of 90-100 preferably 93-95 more preferably 94 amino acids, wherein a third sequence that encodes a first recognition tag is present and has a length of 6-8 amino acids, wherein the fourth sequence that encodes a second recognition tag is present and has a length of 1-10 amino acids, wherein one or more linkers are present and each linker sequence has a length of 1-15 preferably 11 amino acids.

In preferred embodiments the polypeptide comprises
a third sequence that encodes a first recognition tag, and
a fourth sequence that encodes a second recognition tag, wherein the second recognition tag is distinct from the first recognition tag, and preferably
wherein the order of the sequences within the polypeptide, from N-terminus to C-terminus, is either
i) the third sequence, the second sequence, the first sequence, and the fourth sequence; or
ii) the first sequence, the second sequence, the third sequence, and the fourth sequence. This option is most preferred.

In some embodiments the polypeptide comprises a third sequence that encodes a first recognition tag, and a fourth sequence that encodes a second recognition tag, wherein the second recognition tag is distinct from the first recognition tag, and an optional fifth sequence that encodes a third recognition tag, wherein the third recognition tag is distinct from the first and second recognition tags, preferably
wherein the order of the sequences within the polypeptide, from N-terminus to C-terminus, is either
   i) the first sequence, the second sequence, the fourth sequence, and the third sequence; or
   ii) the first sequence, the second sequence, the fifth sequence, the third sequence, and the fourth sequence. This option is most preferred.

In this regard, good results were obtained with the polypeptide wherein the first sequence has the activity of NLuc, the second sequence encodes the A2 domain of VWF, the third sequence is a cysteine residue, the fourth sequence is a histidine-tag, and the fifth sequence is a FLAG-tag.

Preferred polypeptides have at least 95% sequence identity to SEQ ID NOs: 15-18, preferably to SEQ ID NO: 15, 17, or 18, preferably at least 98% sequence identity, most preferably at least 99% or 100% sequence identity such as 100%. More preferred polypeptides have at least 95% sequence identity to SEQ ID NOs: 15-18, 54, or 56, preferably to SEQ ID NO: 15, 17, 18, 54, or 56 preferably at least 98% sequence identity, most preferably at least 99% or 100% sequence identity such as 100%.

### Further Products

The invention provides a nucleic acid construct comprising a sequence that encodes a polypeptide according to the invention. A skilled person knows how to obtain a nucleic acid sequence that encodes any particular polypeptide. Preferably the nucleic acid construct is codon-optimised. The sequence that encodes a polypeptide preferably has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity with any one of SEQ ID NOs: 37, 38, 50, or 51, more preferably at least 90% sequence identity, most preferably at least 95% sequence identity. The sequence that encodes a polypeptide is preferably operably linked to a promoter. Also provided is an expression vector comprising the nucleic acid construct as described above. Also provided is a host cell comprising the expression vector as described above or comprising the nucleic acid construct as described above. Host cells are preferably for multiplication of other products according to the invention, in which case the host cell can be any suitable microbial cell, such as E. coli.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or “expression construct” refer to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. An inducible promoter may also be present but not induced.

Also provided is a device for measuring luminescence, the device comprising a polypeptide according to the invention, preferably wherein the device is a point of care device. Instead of a polypeptide according to the invention, the device can also comprise a nucleic acid construct or a cell as described above, although it is most convenient for the device to directly comprise the polypeptide.

A device according to the invention can for example be a luminometer such as a conventional bench-top luminometer or a luminometer for use in an operation room, or it can be an optical microscope; preferably it is a luminometer. Luminometers and microscopes are known in the art, and a skilled person can select a luminometer or microscope that is suitable for use with the polypeptides according to the invention. The device according to the invention can also be a disposable or non-disposable cartridge or insert or cuvette or reactor volume comprising a compound according to the invention or a combination according to the invention. Such a device is preferably designed for use with a conventional luminometer or microscope.

In preferred embodiments, the device according to the invention is a point of care device. The small volumes that can be measured using the method according to the invention as described later herein make the method ideally suited to point of care analysis, such as bedside analysis, analysis in the field, or analysis while mobile. A preferred point of care device comprises a mobile luminometer and is suitable for measuring enzyme activity in a sample that is obtained or that has been obtained in the field, or while mobile, or at a bedside.

Preferably a device according to the invention does not comprise a light source for use in analysis. Preferably a device according to the invention has multiple volumes or channels that can be used for concurrent analysis of multiple parameters. Preferably, a device according to the invention is configured to analyse a volume of at most 100, 80, 60, 50, 40, 30, 25, 20, 15, 10, 5, 4, 3, 2, or 1 μL of sample, more preferably of at most 15, 10, 5, 4, 3, 2, or 1 μL, even more preferably of at most 5 μL of sample. Preferably a device according to the invention is configured to present its analysis output in real time, such as via a display screen or via a gauge or level indicator.

Polypeptides according to the invention can be physiologically acceptable salts. Such salts are known in the art, and a skilled person can select a suitable salt form. In this context a physiologically acceptable salt is a salt that can still be used as a substrate in assays as exemplified later herein. Examples of physiologically acceptable salts are acid addition salts or alkali salts such as sodium salts or potassium salts. Acid addition salts are preferred. Suitable acid addition salts are salts formed through addition of formic acid, acetic acid, propionic acid, trifluoroacetic acid, mesylic acid, tosylic acid, or hydrohalic acids such as HBr or HCl. In preferred embodiments is provided the compound according to the invention, wherein the compound is an acid addition salt optionally selected from a HCl salt, an acetic acid salt, a formic acid salt, a TFA salt, and a mesylic acid salt, preferably a HCl salt or a TFA salt, most preferably a TFA salt.

### Method

The invention provides a method for quantifying ADAMTS13 in a sample, the method comprising the steps of:
a) contacting the sample with a composition comprising a polypeptide according to the invention to release a polypeptide fragment with luciferase activity; wherein the polypeptide is preferably freely dissolved;
b) separating the polypeptide fragment with luciferase activity from the remainder of the polypeptide according to the invention;
c) contacting the fragment with luciferase activity with a suitable substrate; and
d) determining the relative light intensity generated by the fragment with luciferase activity. Such a method is referred to hereinafter as an assay according to the invention. In this context, quantification of ADAMTS13 can be understood as quantification of the activity of ADAMTS13.

The principle of chemiluminescence involving luciferase is well known by the skilled person. It typically uses luciferase, luciferin, ATP, and molecular oxygen for photon production; Mg²⁺ is known to improve luminescence yield of the reaction. Luciferase catalyzes the conjugation of luciferin to ATP, and also the subsequent oxidation of the luciferyl-AMP intermediate. Ultimately, the luciferase provides an environment in which the oxidized luciferin intermediate rearranges to produce oxyluciferin and a single photon with high-quantum efficiency. Light intensity resulting from such luminescence is dependent on the concentrations of the components involved in the chemical or enzymatic conversion of the liberated luminescent molecule. By using an excess of such components, the luminescent signal of the method of the invention becomes dependent only on the generation of free luminescent molecules by cleavage of a substrate by ADAMTS13. Under such circumstances, the light intensity thus is proportional to the quantity of ADAMTS13, as other components should be present in excess. Thus ADAMTS13 can be quantified through design of the assay according to the invention, as its concentration is proportional to the light output of the assay.

A sample can be a sample from a subject, preferably it is a sample that has been previously obtained from a subject. A subject can be a human. A subject can be non-human. A sample is preferably a fluid. A preferred sample is blood or derived from blood. Suitable samples are whole blood and plasma such as platelet poor plasma or platelet rich plasma. A most preferred sample is platelet poor plasma, such as platelet poor plasma that has been previously obtained from a subject.

Conditions for ADAMTS13 activity are known in the art, for instance using barium chloride or as described by Muia et al. (J Thromb Haemost. 2013 August; 11(8):1511-8. doi: 10.1111/jth.12319), and it is under these circumstances that the contacting is preferably done. An example is the use of a physiologically acceptable buffer, for example Tris buffer optionally comprising 1% serum albumin such as BSA. An example of a suitable Tris buffer is Tris buffered saline (TBS; 50 mM Tris-HCl, 150 mM NaCl; pH 7.4). The concentration of the polypeptide according to the invention is preferably at least 1, 2, 3, 4, 5, 6, 7, 8, 9, 10, 15, 20, 25, 30, 35, 40, 45, or 50 μM or more, more preferably at least 20 or 30 μM such as at least 30 μM. The concentration of the polypeptide according to the invention is preferably at most 5000, 4000, 3000, 2000, 1750, 1500, 1250, 1000, 900, 800, 700, 600, 500, 400, 300, 200, 100, 90, 80, 70, 60 μM or lower, more preferably at most 2500 or 200 μM or 1750 μM or 1500 μM or 1250 μM or 1000 μM or 900 μM or 800 μM or 700 μM or 600 μM or 500 μM or lower, even more preferably at most 2000 or 750 μM or lower, such as at most 750 μM.

Chemiluminescent molecules are known in the art. Examples of chemiluminescent molecules are chemiluminescent amines, which are amines of luciferin such as amines of firefly luciferin, latia luciferin, bacterial luciferin, coelenterazine, cypridinluciferin, or 3-hydroxy hyspidin. The chemiluminescent amine is preferably aminoluciferin of firefly luciferin or optionally a C1-4alkyl ester thereof such as 2-(6-amino-1,3-benzothiazol-2-yl)-4,5-dihydrothiazole-(4/5)-carboxylic acid, more preferably a 2-(6-amino-1,3-benzothiazol-2-yl)-4,5-dihydrothiazole-4-carboxylic acid or optionally a C1-4alkyl ester thereof such as (4S)-2-(6-amino-1,3-benzothiazol-2-yl)-4,5-dihydrothiazole-4-carboxylic acid or optional C1-4alkyl esters thereof, most preferably (4S)-2-(6-amino-1,3-benzothiazol-2-yl)-4,5-dihydrothiazole-4-carboxylic acid.

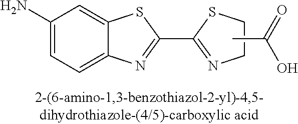

In some embodiments coelenterazine and furimazine and optionally derivatives thereof are preferred substrates. It allows the use of high yield luciferases such as the nanoluc luciferase (SEQ ID NO: 7). In some embodiments luciferin-based substrates and optionally derivatives thereof are preferred substrates. These substrates have improves water solubility and these substrates can be more stable, for instance in plasma. These substrates allow the use of firefly luciferase and related enzymes, which can have improved stability after drying, for instance after drying on a surface.

ATP is required by some types of luciferase and is preferably used in an amount suitable for enabling luciferase activity, or in a stock solution in an amount suitable for preparing dilutions that enable luciferase activity. A suitable ATP stock solution is a 1 mM solution in distilled water, but it can be any stock solution in the range of 200 μM to 10 mM in any physiologically acceptable solvent system.

The method may further comprise use of magnesium ions as it was found that these magnify the luminescent signal generated by luciferase. However, it is also possible to achieve this effect with other divalent cations such as Mn²⁺. An Mg²⁺ source is a source of magnesium ions, which enhances luciferase functioning. Preferred sources of Mg²⁺ are magnesium salts such as magnesium citrate, MgSO₄, MgCO₃, MgO, MgCl₂, MgF₂, MgI₂, MgBr₂, and hydrates thereof. Magnesium halides are more preferred, being MgCl₂, MgF₂, MgI₂, MgBr₂, and hydrates thereof. A most preferred Mg²⁺ source is MgCl₂ or a hydrate thereof.

In step c) the fragment with luciferase activity is contacted with a suitable substrate. The purpose of this contacting is to generate a light quant from a chemiluminescent molecule via the activity of the luciferase domain that was released in step a). Methods for converting a chemiluminescent substrate to produce a light quant are established in the art, and the luciferase, preferably firefly luciferase, can become more functional when further substances are also present during this contacting. Accordingly, in preferred embodiments, step c) further comprises contacting with ATP. In preferred embodiments, step c) further comprises contacting with Mg²⁺. In highly preferred embodiments, step c) further comprises contacting with ATP and Mg²⁺. When ATP is also present during step c), it can be present at a final concentration of about 50-1000 μM, preferably at a final concentration of about 250-500 μM, more preferably of about 300-400 μM such as about 333 μM. When Mg²⁺ is present during step c), it can be present at a final concentration of about 1-30 μM, it is preferably present at a final concentration of about 4-12 mM, more preferably of about 6-10 mM, such as about 8.3 mM. Luciferase is preferably present at about 0.05-50 mg/mL, more preferably at about 0.1-10 mg/mL, even more preferably at about 0.5 to 5 mg/mL such as at about 0.9 mg/mL.

In step d) the luminescent signal is determined. This can be done in any way that is known in the art, for example using a luminometer. The determined light intensity is used as a basis for quantifying ADAMTS13. This is because the concentration of active ADAMTS13 correlates to the relative light intensity, preferably expressed as relative light units (RLU). In some embodiments, the relative light intensity is compared to a reference value or to a calibration curve. Such a calibration curve has preferably been prepared using known amounts of ADAMTS13, for example as demonstrated in the examples. A reference value can be a set value such as a predetermined value, or it can be the assay result from a control sample. In this context a control sample is preferably a sample that is known to meet certain specifications, or a sample (previously) obtained from a healthy subject, or it is normal pooled plasma.

The relative light intensity directly represents the actual luciferase activity, which in turn directly represents ADAMTS13 activity. This is in contrast to accumulating assays such as fluorogenic assays or chromogenic assays. For the latter two assays the slope must be calculated to determine conversion rate. In luminescent assays the flat output (for example in RLU) directly indicates the conversion rate. In preferred embodiments, step d) does not comprise determining a derivative of any signal determined in step d). In more preferred embodiments, step d) does not comprise determining the first derivative of the relative light intensity generated by the luciferase. In this context, light intensity generated by the luciferase relates to the relative light intensity resulting from the luminescent molecule such as aminoluciferin being released from the compound according to the invention. Luminescence can be measured at wavelengths according to methods known in the art. Examples of suitable wavelengths are wavelengths between 360-630 nm.

The relative light intensity as determined at a point in time thus directly provides relevant information about ADAMTS13 activity. Still, determination of relative light intensity during a set duration of time, or until a certain condition is met, can provide relevant information about the dynamics of an assay. Accordingly, in preferred embodiments, step d) comprises determining the relative light intensity generated by the luciferase over a period of time. This period of time is preferably at most 150, 120, 90, 80, 70, 60, 55, 50, 45, 40, 35, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 minute or shorter, more preferably at most 35, 30, 15, or 10 minutes or shorter. A period of time is preferably at least 1, 2, 3, 4, 5, 6, 7, 8, 9, 10, 15, 20, 25, 30, 35, 40, 45, 50, 55, or 60 seconds, more preferably at least 10, 20, or 30 seconds such as at least 30 seconds.

Good results were obtained when the polypeptide according to the invention was immobilized. Accordingly the invention provides the method as described above, the method comprising the steps of:
a) contacting the sample with a composition comprising a polypeptide according to the invention, wherein the polypeptide is immobilized in an assay container, to release a polypeptide fragment with luciferase activity;
b) separating the polypeptide fragment with luciferase activity from the remainder of the polypeptide according to the invention;
c) contacting the fragment with luciferase activity with a suitable substrate and optionally contacting the immobilized polypeptide in the assay container with a suitable substrate; and
d) determining the relative light intensity generated by the fragment with luciferase activity and optionally determining the relative light intensity generated by the immobilized polypeptide in the assay container.

In preferred embodiments the relative light intensity generated by both the separated fragment with luciferase activity and the remaining immobilized polypeptide in the assay container are determined. This can increase the sensitivity of the assay.

Immobilization of the polypeptide can be via any means known in the art. Suitable means of immobilization are via interaction between a recognition tag and a suitable surface, for instance by immobilizing a histidine-tag on a Ni-NTA surface, or by immobilizing a FLAG-tag on an antibody-coated surface, or by immobilizing a cysteine residue on a Michael-acceptor surface such as a maleimide-coated surface. Such surfaces are known in the art and are often commercially available.

The Examples offer various reductions to practice for immobilization of polypeptides according to the invention. Most preferably, when a polypeptide is immobilized, it is immobilized via a cysteine residue in the polypeptide, or via an antibody to the A2-domain of VWF.

Altogether, the present invention provides an improved, sensitive method for monitoring ADAMTS13 or its inhibitors in a test sample. The method of the present invention allows for the design of an optical point-of-care device for measuring the generation of one or more hemostasis factors.

### General Definitions

In preferred embodiments, compounds and compositions according to the invention are for use in methods according to the invention, or are for use according to the invention. Each embodiment as identified herein may be combined together unless otherwise indicated.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

In this document and in its claims, the verb “to comprise” and its conjugations is used in its non-limiting sense to mean that items following the word are included, but items not specifically mentioned are not excluded. “Hemostasis” and “Haemostasis” can be used interchangeably herein. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 1% of the value. In addition, the verb “to consist” may be replaced by “to consist essentially of” meaning that a composition of the invention may comprise additional component(s) than the ones specifically identified, said additional component(s) not altering the unique characteristics of the invention.

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. “Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, MD 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, WI. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A “nucleic acid molecule” or “polynucleotide” (the terms are used interchangeably herein) is represented by a nucleotide sequence. A “polypeptide” is represented by an amino acid sequence. A “polypeptide” as used herein refers to any peptide, oligopeptide, polypeptide, gene product, expression product, or protein. A polypeptide is comprised of consecutive amino acids.

The term “polypeptide” encompasses naturally occurring and synthetic molecules.

All patent and literature references cited in the present specification are hereby incorporated by reference in their entirety.

In the context of this invention, a cell or a sample can be a cell or a sample from a sample obtained from a subject. Such an obtained sample can be a sample that has been previously obtained from a subject. Such a sample can be obtained from a human subject. Such a sample can be obtained from a non-human subject.

### PARTICULAR EMBODIMENTS

1. Polypeptide comprising a first sequence and a second sequence, wherein the first sequence encodes a polypeptide that has luciferase activity, and wherein the second sequence encodes a von Willebrand factor (VWF) domain that comprises a tyrosine-methionine recognition site for ADAMTS13.
2. The polypeptide according to embodiment 1, wherein the first sequence encodes a polypeptide that has the activity of Enhanced Beetle Luciferase (ELuc), Click Beetle Green Luciferase (CBG), R. ohbai Luciferase (RoLuc), Firefly Luciferase (FLuc), Red Firefly Luciferase (RedF), P. hirtus Red Luciferase (RedLuc), Nano Luciferase (NLuc), Renilla Luciferase (Renilla), Metridia Luciferase (MetLuc), Lucia Luciferase (Lucia) Gaussia Luciferase (GLuc), or Green Renilla Luciferase (GrRenilla), preferably it has the activity of NLuc.
3. The polypeptide according to embodiment 1 or 2, wherein the first sequence has at least 70% sequence identity with any one of SEQ ID NOs: 1-12, preferably with SEQ ID NO: 7, preferably at least 90% sequence identity, more preferably at least 98% sequence identity.
4. The polypeptide according to any one of embodiments 1-3, wherein the second sequence has at least 70% sequence identity with any one of SEQ ID NOs: 13-14, preferably with SEQ ID NO: 14, preferably at least 90% sequence identity, more preferably at least 98% sequence identity.
5. The polypeptide according to any one of embodiments 1-4, wherein the second sequence encodes the A2 domain of VWF, wherein the tyrosine-methionine recognition site for ADAMTS13 comprises a tyrosine at position 105, 106, 107, 108, 109, 110, or 111, wherein that tyrosine is directly followed by a methionine, optionally wherein the tyrosine-methionine recognition site is directly flanked by two valine residues.
6. The polypeptide according to any one of embodiments 1-5, wherein the first sequence is N-terminal to the second sequence, or wherein the second sequence is N-terminal to the first sequence, preferably wherein the second sequence is N-terminal to the first sequence.
7. The polypeptide according to any one of embodiments 1-6, wherein the polypeptide comprises a third sequence that encodes a first recognition tag, and optionally comprises a fourth sequence that encodes a second recognition tag, wherein the second recognition tag is distinct from the first recognition tag.
8. The polypeptide according to embodiment 7, wherein the recognition tag is an epitope tag, an affinity-tag, a cysteine residue, or an aptamer-tag, preferably the recognition tag is a histidine-tag or a FLAG-tag or a cysteine residue.
9. The polypeptide according to any one of embodiments 1-8, wherein the first sequence and the second sequence are separated by a linker sequence, wherein the linker sequence comprises 1-40 amino acids, preferably 3-30 amino acids.
10. The polypeptide according to any one of embodiments 1-9, wherein the polypeptide comprises 200-500 amino acids, preferably 250-350 amino acids, more preferably 265-325 amino acids, optionally wherein the first sequence has a length of 150-450 amino acids, optionally wherein the second sequence has a length of 50-200 amino acids, optionally wherein a third sequence that encodes a first recognition tag is present and has a length of 4-25 amino acids, optionally wherein the fourth sequence that encodes a second recognition tag is present and has a length of 4-25 amino acids, optionally wherein one or more linkers are present and each linker sequence has a length of 1-30 amino acids.
11. The polypeptide according to any one of embodiments 1-10, wherein the polypeptide comprises a third sequence that encodes a first recognition tag, and a fourth sequence that encodes a second recognition tag, wherein the second recognition tag is distinct from the first recognition tag, and preferably wherein the order of the sequences within the polypeptide, from N-terminus to C-terminus, is either i) the third sequence, the second sequence, the first sequence, and the fourth sequence; or ii) the first sequence, the second sequence, the third sequence, and the fourth sequence.
12. The polypeptide according to any one of embodiments 1-11, wherein the polypeptide has at least 95% sequence identity to SEQ ID NOs: 15-18, optionally SEQ ID NOs: 54 or 56, preferably to SEQ ID NO: 15, 17, or 18, preferably at least 98% sequence identity.
13. Nucleic acid construct comprising a sequence that encodes a polypeptide as defined in any one of embodiments 1-12.
14. Device for measuring luminescence, the device comprising a polypeptide as defined in any one of embodiments 1-12, preferably wherein the device is a point of care device.
15. Method for quantifying ADAMTS13 in a sample, the method comprising the steps of: a) contacting the sample with a composition comprising a polypeptide as defined in any one of embodiments 1-12 to release a polypeptide fragment with luciferase activity; wherein the polypeptide is preferably freely dissolved; b) separating the polypeptide fragment with luciferase activity from the remainder of the polypeptide as defined in any one of embodiments 1-12; c) contacting the fragment with luciferase activity with a suitable substrate; and d) determining the relative light intensity generated by the fragment with luciferase activity.
16. The polypeptide according to any one of embodiments 1-12, wherein the first sequence does not comprise a cysteine residue, preferably wherein it has at least 70% sequence identity with SEQ ID NO: 39, preferably at least 90% sequence identity, more preferably at least 98% sequence identity.
17. The polypeptide according to embodiment 16, wherein the polypeptide comprises a third sequence that encodes a first recognition tag, wherein the first recognition tag is a cysteine residue.
18. The polypeptide according to embodiment 17, wherein the polypeptide comprises a fourth sequence that encodes a second recognition tag, wherein the second recognition tag is distinct from the first recognition tag.
19. The polypeptide according to embodiment 18, wherein the polypeptide comprises a third sequence that encodes a first recognition tag, and a fourth sequence that encodes a second recognition tag, wherein the second recognition tag is distinct from the first recognition tag, and an optional fifth sequence that encodes a third recognition tag, wherein the third recognition tag is distinct from the first and second recognition tags, preferably wherein the order of the sequences within the polypeptide, from N-terminus to C-terminus, is either i) the first sequence, the second sequence, the fourth sequence, and the third sequence; or ii) the first sequence, the second sequence, the fifth sequence, the third sequence, and the fourth sequence.
20. The polypeptide according to embodiment 19, wherein the first sequence has the activity of NLuc, the second sequence encodes the A2 domain of VWF, the third sequence is a cysteine residue, the fourth sequence is a histidine-tag, and the fifth sequence is a FLAG-tag.
21. The method according to embodiment 15, the method comprising the steps of: a) contacting the sample with a composition comprising a polypeptide as defined in any one of embodiments 1-12 or 16-20, wherein the polypeptide is immobilized in an assay container, to release a polypeptide fragment with luciferase activity; b) separating the polypeptide fragment with luciferase activity from the remainder of the polypeptide as defined in any one of embodiments 1-12; c) contacting the fragment with luciferase activity with a suitable substrate and optionally contacting the immobilized polypeptide in the assay container with a suitable substrate; and d) determining the relative light intensity generated by the fragment with luciferase activity and optionally determining the relative light intensity generated by the immobilized polypeptide in the assay container.

### DESCRIPTION OF DRAWINGS

FIG. 1—The concentration of ADAMTS13 increases when the NPP concentration increases. For the fusion protein of SEQ ID NO: 15 this results in a decreasing signal because of the cleavage by ADAMTS13. The fusion protein of SEQ ID NO: 16 shows less effect. This fusion protein is also cleaved by ADAMTS13 but here is not as much influence on light emission. Legend indicates SEQ ID NOs.

FIG. 2—Linearity of dilutions is tested with a x-axis in log. The line is plotted with a nonlinear fit analysis.

FIG. 3A—Recovery of the luminescent ADAMTS13 assay versus the reference Technoclone kit (=100%). 15 samples included for the total EP10 analysis. Plasma calibration is to the left of r13A. Data points are expressed as mean±SD.

FIG. 3B—Bias of the luminescent ADAMTS13 assay versus the reference Technoclone kit (=100%). 15 samples included for the total EP10 analysis. Plasma calibration is to the left of r13A. Data points are expressed as mean±SD.

FIG. 4—Schematic representation of antibody-mediated immobilization of polypeptides of the invention. Polypeptide is represented by a series of blocks, where the block without a label represents the VWF-domain (A13-cleavage site indicated). After cleavage, the luciferase domain either remains in the binding plate or can be transferred to a transfer plate.

FIG. 5A—Schematic representation of cysteine-mediated immobilization of polypeptides of the invention. When cut, the luciferase domain can enter solution and be washed away. Uncut polypeptide will lead to a signal on the plate itself.

FIG. 5B—Calibration curve of remaining polypeptide on the binding plate.

FIG. 5C—Signal as a function of A13 concentration.

FIG. 5D—Sensitivity can be increased when both the remaining (binding) polypeptide as well as the transferred (solution) polypeptide are measured.

FIG. 6—SDS-PAGE gel (4-15% gradient) stained with Coomassie Blue. Shown are polypeptide according to the invention, and its fragments after A13 cleavage.

FIG. 7A—Development of a benchtop assay to demonstrate validity of the A13 activity assay. Maximum binding capacity of the Nluc-VWFA2-FLAG-His fusion protein to 1000 ng/well monoclonal mouse M2 FLAG capture antibody (measured in a transparent plate in sixfold).

FIG. 7B—as 7A, but A13 activity assay in which the cleaved-off A13 is transferred to a new plate (measured in a white luminescence plate in quadruplicate).

FIG. 8A—Development of a Ni-NTA immobilized A13 activity benchtop assay. Binding of the VWF-A2 fusion protein to the Ni²⁺-NTA plate.

FIG. 8B—Transfer of released VWF-A2 fusion protein after A13 digestion.

FIG. 8C—Calibration curve of remaining VWF-A2 fusion protein on the binding plate (measured in quadruplicate).

FIG. 9—Comparison between two analogues of SEQ ID NO: 17 where the Nluc part was replaced by either one of two different firefly luciferases (SEQ ID NOs as indicated).

FIG. 10—SDS-page gel (4-15% gradient) stained with Coomassie Blue shows the expected fragments of the fusion-protein (SEQ ID NO: 17) after cleavage by adding ADAMTS13.

FIG. 11—NPP sowed a significantly higher signal output compared to ADAMTS13 deficient plasma when allowed to liberate a luciferase which is then used in a chemiluminescent assay.

### EXAMPLES

### Example 1—Overview of Luminescent-Based ADAMTS13 Activity Assay

Background: The activity of Von Willebrand Factor (VWF) is modulated by ADAMTS13, a metalloprotease that cleaves VWF in the A2 domain. Deficiency in ADAMTS13 results in the disorder thrombotic thrombocytopenia purpura (TTP). ADAMTS13 activity analysis for initial diagnosis of TTP is the first-tier assay recommended by the ISTH in 2020. A point-of-care ADAMTS13 test could aid in the therapeutic decision-making process.

Aims: To develop a sensitive luminescent ADAMTS13 activity assay for a point of care platform.

Methods: A chimeric protein construct was expressed in BL21 E. coli containing the VWFA2 domain (SEQ ID NO: 14) (with the ADAMTS13 cleavage site Y1605-M1606) in frame with N-terminal NanoLuc® Luciferase (SEQ ID NO: 7), a C-terminal His-tag, and a free cysteine at the C-terminus to enable purification and immobilization, respectively. This construct (N-NLuc-VWFA2-C, SEQ ID NO: 17) was purified and shown to be cleaved by ADAMTS13 by SDS-PAGE in the expected fragments. N-NLuc-VWFA2-C coupled via the free cysteine to commercially available maleimide-coated plates was also cleaved by ADAMTS13 as shown by the released luciferase activity in the supernatant transferred to wells with furimazine substrate. Titration of recombinant ADAMTS13 in ADAMTS13-deficient plasma was performed to verify sensitivity towards ADAMTS13 activity levels in plasma. The luminescent ADAMTS13 activity assay (ADAMTS13lum) was compared to the TECHNOZYM® ADAMTS13 Activity ELISA from Technoclone, using samples from ECAT surveys.

Results: Titration of rADAMTS13 (TAK 755, Takeda) in deficient plasma showed excellent concentration dependent luminescence of the ADAMTS13lum assay over the range of 0-2 IU/ml (R² 0.997). Estimation of ADAMTS13 activity levels of 4 ECAT samples with a range between 0.07 to 0.82 IU/ml with the ADAMTS13lum assay showed high level of agreement (deviance<6%) with the levels estimated by TECHNOZYM® ADAMTS13 Activity ELISA. Constructs using various firefly luciferases instead of NanoLuc also showed substrate-dependent response, demonstrating the modularity of the construct design.

Conclusion: A luminescent-based assay specific for ADAMTS13 activity was developed. Because of the high sensitivity of this technique, it is suitable for further miniaturization to a point-of-care platform and small plasma volumes.

### Example 2—Detailed Luminescent-Based ADAMTS13 Activity Assay

### 2.1 Introduction

For a chemiluminescent ADAMTS13 activity assay, two polypeptides were expressed and analyzed. Both polypeptides can be seen as fusion proteins and comprise the A2 domain (SEQ ID NO: 14) with the residues aa1574-1668 of the VWF molecule, a His-tag (here SEQ ID NO: 19), a Flag-tag (here SEQ ID NO: 24), and a Nanoluc luciferase (SEQ ID NO: 7). The A2 domain binds to a relevant antibody and can be recognized and cleaved by ADAMTS13. The His-tag is included into the design for the purification step of the fusion protein. The Nanoluc luciferase (Nluc) catalyzes the production of photons in the presence of coelenterazine or furimazine, resulting in a quantifiable signal. The difference between the two fusion proteins is the sequence of the domains. One fusion proteins contains the Nluc at the N-terminus (SEQ ID NO: 15) while the second fusion protein contains the Nanoluc at the C-terminus (SEQ ID NO: 16). For both polypeptides the His-tag is C-terminal. Both fusion proteins were analyzed.

Additional designs based on SEQ ID NO: 15 were produced having luciferases at the N-terminus, for instance featuring Nluc (resulting in the construct of SEQ ID NO: 17), optimised P. photuris firefly luciferase also known as YY5 (the luciferase being represented by SEQ ID NO: 53, the fusion protein by SEQ ID NO: 54), or optimised P. pennsylvanica firefly luciferase also known as Luc90 (the luciferase being SEQ ID NO: 55, the fusion protein by SEQ ID NO: 56).

The chemiluminescent assay design uses an ELISA plate coated with an antibody against the A2 domain. The sample and the fusion protein are added to these coated wells, resulting in the cleavage of the fusion protein by the ADAMTS13 that is present in the sample. It depends on which part of the fusion protein is cleaved and removed from the well during the washing step if the reaction results in a signal or not. When the NanoLuc is still present in the well, photons will be generated when adding furimazine or coelenterazine. It was found that when the fusion proteins are cleaved by ADAMTS13 into 2 new products, one of these products, containing section aa1606-1668 of the A2 domain of VWF, still binds to the antibodies. This means that whether or not the fusion protein is cleaved, there will always be the same signal. When Nluc instead is released and removed via washing, the signal will decrease when more fusion protein is cleaved.

### 2.2 Reagents

| Reagent | References |
|---|---|
| Goat-α-human VWF | Affinity Biologicals, lot# AP3651-AR1 |
| Mouse-α-human VWF-A2 | R&D Systems, lot# JKX0219091, clone: 210909 |
| HEPES buffer | 25 mM HEPES, 125 mM NaCl, 10 mM CaCl |
| Coelenterazine | Carbosynth Ltd, lot#0000038752, ref: EC181113 |
| Normal pooled plasma (NPP) | ref nr. LHNP12062017 (platelet poor plasma) |
| Furimazine | Promega, lot#0000473010, ref: N113B |
| Wilfactin | LFB, product# 13L06795, RVG# 32375, |
| rADAMTS13 | Baxter, lot# HR5AR00, Art# 1504182 |
| Heparin tubes | BD vacutainer, lot# 0058541, ref#368884 |
| Proclin 300 | Sigma-aldrich, lot# MKCN8358 |

### 2.3.1 Antibody Coating

For antibody coating, a 96-well, transparent, high-binding ELISA plate from Corning (#3590) was used. The wells were coated with 500 ng/well of affinity purified polyclonal capture antibody goat-a-human VWF or 500 ng/well of the monoclonal antibody Mouse-a-human VWF-A2 overnight at room temperature (RT) in HEPES. Subsequently, the wells were blocked with 1% BSA blocking solution and washed using 0.1% Tween (Tw-HEPES).

### 2.3.2 Fusion Protein Affinity Test

The 96-well plate was coated with polyclonal goat-a-human VWF and monoclonal mouse-a-human VWF-A2 antibodies. Afterwards, the wells were incubated with HEPES-BSA buffer containing between 0 and 1000 ng/well of the fusion proteins for 1 h at RT. After the final washing step is executed, 100 μl of 170 μM coelenterazine in HEPES was added to the wells. The signal was measured using SpectraMax M3 plate reader (Molecular Devices) in luminescence mode. Luminescence was measured (RLU) every 0.5 min. for 30 min. for 200 mS.

### 2.3.3 Fusion Proteins Cleavage Ability

The 96-well plate was coated with monoclonal mouse-a-human VWF-A2 antibodies as mentioned above. Afterwards the wells were incubated with PBS-BSA buffer containing 200 ng/well of the fusion proteins and 0-20% NPP for 1 h at RT. Ultimately, after extensive washing, 100 μl of 170μM coelenterazine in HEPES buffer was added to the wells and the plate was immediately transferred to the SpectraMax M3 plate reader (Molecular Devices) in luminescence mode. Luminescence was measured (RLU) every 0.5 min. for 30 min. for 200 mS.

### 2.3.4 Competition Between VWF and the Fusion Proteins

The 96-well plate was coated with monoclonal mouse-a-human VWF-A2 antibodies as described above. Afterwards the wells were incubated with PBS-BSA buffer containing 200 ng/well of SEQ ID NO: 15 and 0-1.0 IU/ml Wilfactin™ for 1 h at RT. Ultimately, after extensive washing, 100 μl of 170 μM coelenterazine in HEPES was added to the wells and the plate was immediately transferred to the SpectraMax M3 plate reader (Molecular Devices) in luminescence mode. Luminescence was measured (RLU) every 0.5 min. for 30 min. for 200 mS.

### 2.3.5 ADAMTS13 Titration

Anti-ADAMTS13 (I1-1 auto-antibodies) were added to NPP and 1 IU/ml rADAMTS13 in amounts of 0-5 μg/ml. Subsequent the fusion protein represented by SEQ ID NO: 15 was added, and the mixtures were incubated 2 h at 37° C.

For this experiment, the 96-well plate was coated with monoclonal mouse-a-human VWF-A2 antibodies as described above. Afterwards the wells were incubated with the mixtures for 1 h at RT. Ultimately, after final washing, 100 μl of 400× diluted furimazine in HEPES was added to the wells and the plate was immediately transferred to the SpectraMax M3 plate reader (Molecular Devices) in luminescence mode. Luminescence was measured (RLU) every 0.5 min. for 10 min. for 200 mS.

### 2.3.6 Fusion Protein Optimization

For this experiment, the 96-well plate was coated with monoclonal mouse-a-human VWF-A2 antibodies as described above but with 250 ng/well. Afterwards the wells were incubated with HEPES-BSA buffer containing amounts of 200-100-50-25-12.5-6.25-3.125-1.56 and 0 ng/well of SEQ ID NO: 15 and 0-0.05-0.1-0.2-0.5-1.0 IU/ml rADAMTS13 1 h at RT. Ultimately, after extensive washing, 100 μl of 400× diluted furimazine in HEPES was added to the wells and the plate was immediately transferred to the SpectraMax M3 plate reader (Molecular Devices) in luminescence mode. Luminescence was measured (RLU) every 0.5 min. for 10 min. for 200 mS.

### 2.3.7 Dilution/Dynamic Range Optimization

For this experiment, the 96-well plate was coated with monoclonal mouse-a-human VWF-A2 antibodies but with 250 ng/well. NPP was diluted in BSA-HEPES dilution in concentrations of 0-0.05-0.1-0.2-0.5-1 IU/ml ADAMTS13. These were then diluted 1,1,5 and 1.15 final concentrations. Afterwards the wells were incubated with 0-0.05-0.1-0.2-0.5-1 IU/ml NPP and 12.5 ng/well of SEQ ID NO: 15 1 h at RT. Ultimately, after extensive washing, 100 μl of 400× diluted furimazine in HEPES was added to the wells and the plate was immediately transferred to the SpectraMax M3 plate reader (Molecular Devices) in luminescence mode. Luminescence was measured (RLU) every 0.5 min. for 10 min. for 200 mS.

### 2.3.8 EP10 Protocol

Before EP10, multiple preparations were done like collecting heparin plasma, making the high, mid and low sample, making calibration samples, measure the high, mid, low, 100% recombinant ADAMTS13 and 100% heparin plasma samples with the technoclone kit.

Preparation of the reagents: Heparin plasma was collected by drawing blood (collected from an inventor) in heparin tubes and centrifuge the tubes at 3000 RCF for 5 min. The heparin plasma was then transferred in new epps. The high sample was made from 80% heparin plasma and 20% ADAMTS13 deficient plasma. For the low 6% was heparin plasma and 94% was ADAMTS13 deficient plasma. The mid sample was made with 50% high sample and 50% low sample. For the EP10 were two calibration lines used, one made with recombinant ADAMTS13+ADAMTS13 deficient plasma and one with heparin plasma+ADAMTS13 deficient plasma. The for the calibration line with recombinant ADAMTS13 was the ADAMTS13 first diluted 1:300 in ADAMTS13 deficient plasma to create a 100% sample. This sample was diluted till the concentrations of 50-25-12.5-6.25-3.125%. For the calibration line with heparin plasma was the plasma used as 100% sample and these was diluted till the concentrations of 50-25-12,5-6,25-3,125%. For this experiment, 96-well plates coated monoclonal mouse-a-human VWF-A2 antibodies but with 250 ng/well. The blocking buffer used this time also contains 0.05% proclin 300. When the plate was blocked, these were stored in the fridge at 4° C. The fusion protein having SEQ ID NO: 15 was diluted till a concentration of 12.5 ng/well in 1% BSA for the hole protocol.

Technoclone Technozyme kit: The Technoclone kit is a commercially available ELISA for measuring ADAMTS13 activity in human plasma and is used to measure the true values of the samples and the calibration curves. The technoclone kit contains ELISA strips which are coated with anti-GST antibodies, lyophilized GST-vWF73 substrate, lyophilized calibration plasma with 6 different concentration for a calibration line, lyophilized control plasma high and low, reaction buffer, conjugate monoclonal HRP labeled antibody, Tetramethylbenzidine (TMB) substrate, wash buffer 10× concentrated, stop liquid containing sulfuric acid 2.5% and a microtiter plate for sample dilution. Before use, the lyophilized GST-vWF73 substrate, lyophilized calibration plasma, lyophilized control plasma and the wash buffer need preparations. The GST-vWF73 substrate was dissolved in 6 ml MiliQ, lyophilized calibration plasma and lyophilized control plasma was dissolved in 500 μl MiliQ and the wash buffer was 1:9 diluted in MiliQ. The samples were diluted 1:30 in the reaction solution of the kit.

| Step description | Implementation step of the provision |
|---|---|
| GST-vWF substrate incubation | 100 μl per well for 1 hour at room temperature |
| Wash with wash buffer | 3 × 300 μl per well |
| Sample incubation | 100 μl per well for 30 minutes at room |
| | temperature |
| Wash with wash buffer | 3 × 300 μl per well |
| Conjugate incubation | 100 μl per well for 1 hour at room temperature |
| Wash with wash buffer | 3 × 300 μl per well |
| TMB color substrate reaction | 100 μl per well for 30 minutes at room |
| | temperature |
| Stop reaction | 100 μl |
| Measuremet absorbance at 450 mS | 5 seconds with pre-shake within 10 min after |
| | stop reaction |

The assay protocol: For this experiment washing solution was prepared with HEPES buffer and 0.1% Tween, furimazine was diluted 400× in HEPES buffer. The 96-well plate was coated with monoclonal mouse-a-human VWF-A2 antibodies as mentioned above but with 250 ng/well.

| Step description | Implementation step of the provision |
|---|---|
| Wash blocked plate with wash solution | 3 × 300 μl per well |
| Adding samples to the wells | 33.3 μl sample per well |
| Adding fusion protein solution | 66.6 μl solution per well |
| Incubation sample + fusion protein | 1 hour at room temperature |
| Wash with wash solution | 3 × 300 μl per well |
| Adding furimazine | 100 μl per well |
| Measuring the luminescence | 10 minutes with an interval of 30 seconds and |
| | measurements of 200 mS |

EP10 analysis: The raw data gained from the SpectraMax M3 plate reader (Molecular Devices) was edited with GraphPad Prism 9. For the calibration lines, a 4PL (4 parameter logistic) fit was used to inter- and extrapolate the values of the samples. The 4PL fit was chosen because it is commonly used by ELISA assays. The measured data from the calibration lines was used for the EP10 analysis. In this analysis the Bias and the Combined Variation coefficient (CV %) are important values. The Bias shows the difference between the observed measured concentration and the reference measured concentrations of the samples. This means that the Bias needs to be as low as possible because this shows the difference between the reference method and the tested method. This also goes for the CV % but parameter targets for a value under 20% and between 20% and 30% is also acceptable. A CV % higher as 30% needs to be rejected or evaluated. The grand mean is the mean of al the measured samples of all days that are used for EP10.

| Definition | Formula |
|---|---|
| V_{w1} = within-run variance | Within-run SD² |
| V_{w2} = pooled within-run variance | Mean V_{w1} |
| Vₘ = variance of daily means | (SD of all means)² |
| V_{d} = adjusted between-day- variance | (Vₘ) − (V_{w2})/3 |
| Vₜ = combined variance | (V_{w2}) + (V_{d}) |
| S = combined standard deviation | √(Vₜ) |
| Y = grand mean | Mean of all means |
| T = reference values | 0.21-0.62 and 1.08 IU/ml |
| D = Bias | (Y) − (T) |
| C = combined variance coefficient (CV %) | (S/Y) * 100% |

All data of EP10 was tested with the Grubbs test for any outliers. If an outlier was established, this will be called at the results and potentially rejected if necessary. The significance of all the days will be calculated by preforming a one sample sign test. In this test each parameter is estimate value had given a negative or positive value. If all the value of from the parameter are the same sign, then the sign test was significant at the p=0.06 level.

### 2.3.9 Comparison of Different Types of Luciferases

Preparation of the reagents: the calibration curve was prepared by spiking recombinant ADAMTS13 (TAK 755, Takeda) in ADAMTS13 deficient plasma. The calibration samples contained ADAMTS3 concentrations of 2-1-0.5-0 IU/mL. For this experiment, 96-well plates coated with maleimide were used. The fusion proteins were diluted till a concentration of 100 nM in buffer containing 0.1 M sodium phosphate, 0.15 M sodium chloride and 10 mM EDTA (pH 7.2).

### Assay Protocol:

| Step description | Implementation step of the provision |
|---|---|
| Coat 96-well plate with 100 nM fusion protein | 100 μL per well |
| Incubation step 1 | 2 hours at RT |
| Wash with 0.1M Sodium phosphate, 0.15M | 3 × 300 μL per well |
| Sodium chloride and 0.1% Tween | |
| Blocking solution containing 2 mM | 200 μL per well |
| D-Cysteine in 1× PBS (0.1M Na2HPO4, | |
| 0.018M KH2PO4, 1.37M NaCl, 0.027M KCl, pH | |
| 7.4). | |
| Incubation step 2 | 1 hour at RT |
| Wash with 0.1M Sodium phosphate, 0.15M | 3 × 300 μL per well |
| Sodium chloride and 0.1% Tween | |
| Blocking solution containing 1% BSA in 1× PBS | 200 μL per well |
| (0.1M Na2HPO4, 0.018M KH2PO4, 1.37M | |
| NaCl, 0.027M KCl, pH 7.4). | |
| Incubation step 3 | 1 hour at RT |
| Wash with 0.1M Sodium phosphate, 0.15M | 3 × 300 μL per well |
| Sodium chloride and 0.1% Tween | |
| Add activation buffer: 10 mM CaCl₂, 25 mM | 50 μL per well |
| HEPES, 125 mM NaCl, pH 7.4 | |
| Add prediluted samples: the samples are 7.5 | 50 μL per well |
| times prediluted in 57 IU/mL heparin buffer | |
| (0.5% BSA, 25 mM HEPES, 125 mM NaCl, | |
| pH 7.4). | |
| Incubation step 4 | 1 hour at RT |
| Transfer supernatant to new white 96-well plate | 75 μL per well |
| Add furimazine substrate to wells treated with | 25 μL per well |
| NanoLuc fusion protein: Furimazine dissolved | |
| in ethanol was 50 times diluted in HEPES buffer | |
| to 40 μg/mL. | |
| Add luciferin substrate to wells treated with | 25 μL per well |
| firefly luciferases fusion protein: Luciferin [2 | |
| mM], ATP [8 mM], MgCl2 [10 mM] were diluted | |
| in 0.5% BSA, 25 mM HEPES, 125 mM NaCl, | |
| pH 7.4. | |
| Measuring the luminescence | 10 minutes with an interval of 30 seconds and |
| | measurements of 200 mS. |

### 2.4 Results

### 2.4.1 Fusion Protein Binding Ability

The fusion proteins were tested for the binding capacity to the monoclonal VWF-A2 antibody and the polyclonal VWF antibody. If the fusion protein can bind to the antibodies, the signal will increase with the concentration the fusion protein. If the fusion protein is not able to bind an antibody, the signal will be equal to the blank. It was found that he binding capacity of both fusion proteins is low for the polyclonal VWF. The signals were much higher when binding with the monoclonal VWF-A2.

### 2.4.2 Fusion Protein Cleavage Ability

The fusion proteins must be cleaved by ADAMTS13 to be qualified for the ADAMTS13 assay. Cleavage of the fusion protein should result in the release of the NanoLuc, meaning that the increase of the ADAMTS13 concentrations correlates to a decreased signal. The experiment was performed with NPP containing healthy levels of ADAMTS13.

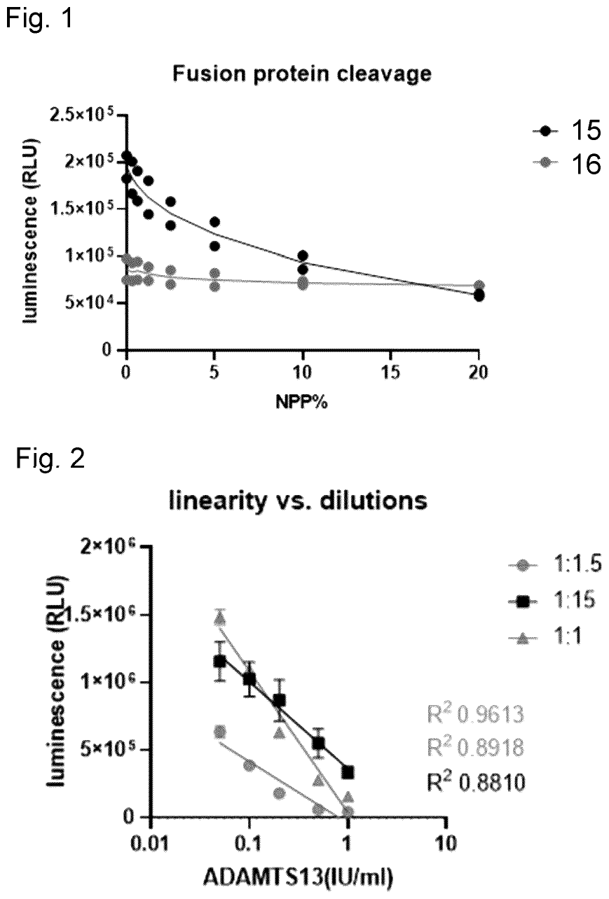

The fusion protein of SEQ ID NO: 15 shows a decreasing signal when the concentration NPP increases, suggesting that ADAMTS13 cleaves the fusion protein. SEQ ID NO: 16 did not demonstrate an effect of an increasing ADAMTS13 concentration (FIG. 1).

### 2.4.3 Wilfactin (VWF) and Fusion Protein Competition

The fusion protein contains the A2 domain of VWF. This could mean that VWF present in plasma can compete with the fusion protein for the binding to the antibodies, resulting in a matrix effect. If the fusion protein competes with VWF, the signal will decrease when the concentration VWF increases. If there is no competition the signal will be unaffected. Wilfactin is used to analyze the competition between the fusion protein and VWF. It was found that the fusion proteins were unaffected by the VWF concentration, implying no competition with VWF.

### 2.4.4 ADAMTS13 Titration

Decreased levels of ADAMTS13 can be caused by deficiency (cTTP) or mostly by autoantibodies (iTTP). It is therefore interesting to examine the effect of antibodies in the ADAMTS13 assay. By using a constant level of ADAMTS13 (from NPP or recombinant ADAMTS13) the activity can be inhibited by autoantibodies against ADAMTS13 (anti-ADAMTS13). If the anti-ADAMTS13 inhibits the ADAMTS13 activity, the fusion protein cannot be cleaved resulting in an increase in signal. It was found that SEQ ID NO: 15 shows an increasing line when the concentration anti-ADAMTS13 increases. SEQ ID NO: 16 does not show an effect with varying anti-ADAMTS13 concentrations.

### 2.4.5 Fusion Protein Optimization

By optimization of the fusion protein concentration the waste of this protein can be minimized and the dynamic range of the assay can be enlarged. The optimization of the fusion protein concentration was done by determining the biggest dynamic range per concentration. If the dynamic range is bigger, it is easier to determine a concentration ADAMTS13 because of the bigger differences between each concentration. It was found that for the dynamic range of different concentrations fusion protein between 200 and 1,56 ng/well, the concentration 12.5 ng/well has the biggest dynamic range.

### 2.4.6 Dilution/Dynamic Range Visualization

The dilution of the sample can be important because a higher dilution means a lower chance of matrix effect. It is also important that the line is linear because these lines are easier to use for determining the concentration of a sample. A steeper line has also the preference because the sensitivity of the assay increases. Three dilutions were tested in this experiment and shown in FIG. 2. The choice of the 1:1.5 and the 1:15 dilutions were based on the fact that these dilutions are often used by various tests. The 1:1 dilution is the dilution that is used in the process of the development of this assay. To decide which dilution is the best, it is important to look for the best linear fit. The 1:15 dilution shows a R² of 0.8810 which is the lowest. The line shows a good fit in the points but a big variety. The 1:1.5 dilution shows the lowest R² of 0.8918 and in the line, it is seeming that the higher concentration, 1 IU/ml, is less linear as the lower concentrations. The best linear fit is the 1:1 dilution. This one has a R² of 0.9613 and the slope is steeper.

### 2.4.7.1.1. Evaluation Protocol 10

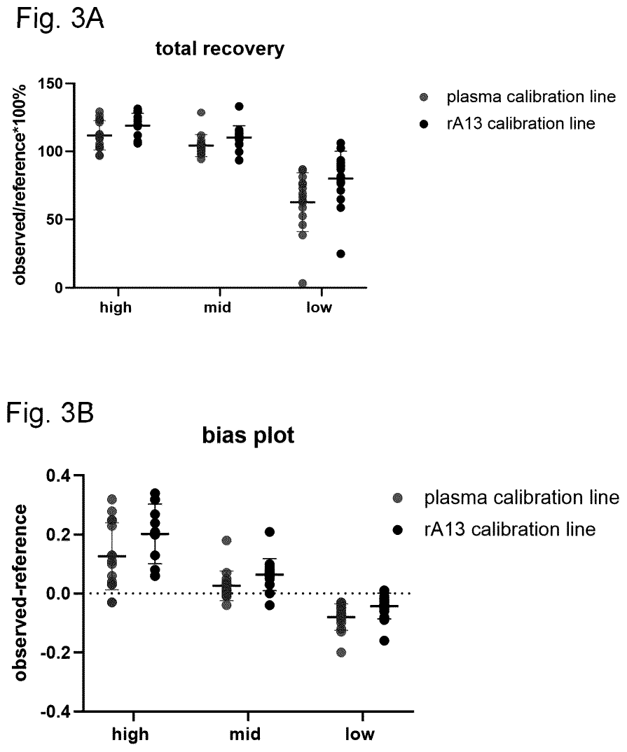

For EP10 the concentration ADAMTS13 in the samples were measured and used as standard. Calibration curves for heparin plasma (R²=0.9552) and recombinant ADAMTS13 (rADAMTS13, R2=0.9696) were similar, as were linear regression (heparin R²=0.9839, rADAMTS13 R²=0.9894). Total recovery and bias plots showed good results (FIG. 3).

### 2.4.8 Comparison of Different Types of Luciferases

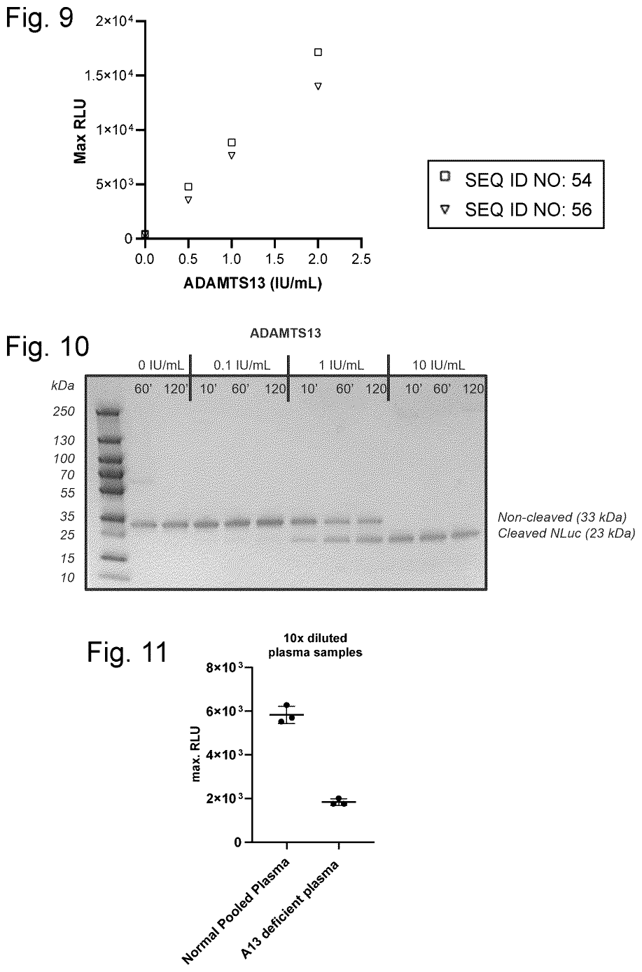

FIG. 9 shows a comparison between the two fusion proteins represented by SEQ ID NOs: 54 and 56, thus being versions of SEQ ID NO: 17 where NanoLuc was replaced by different types of firefly luciferase. The two firefly luciferases show minimal differences, and the signal output is dependent on the amount of ADAMTS13 present in the sample. NanoLuc had a substantially higher luminescent signal output and also showed dose/response relative to ADAMTS13. Therefore, it can be concluded that all three fusion-proteins can be used for luminescence-based ADAMTS13 quantification assays.

### 2.5 Conclusion

Polypeptides according to the invention can reliably be used in ADAMTS13 assays. For polypeptides where the luciferase domain is N-terminal immobilisation could be effected via antibody binding, preferably monoclonal antibody binding. The universal applicability of the polypeptide design was demonstrated using multiple different luciferase enzymes.

### Example 3—Different Strategies for Polypeptide Immobilisation

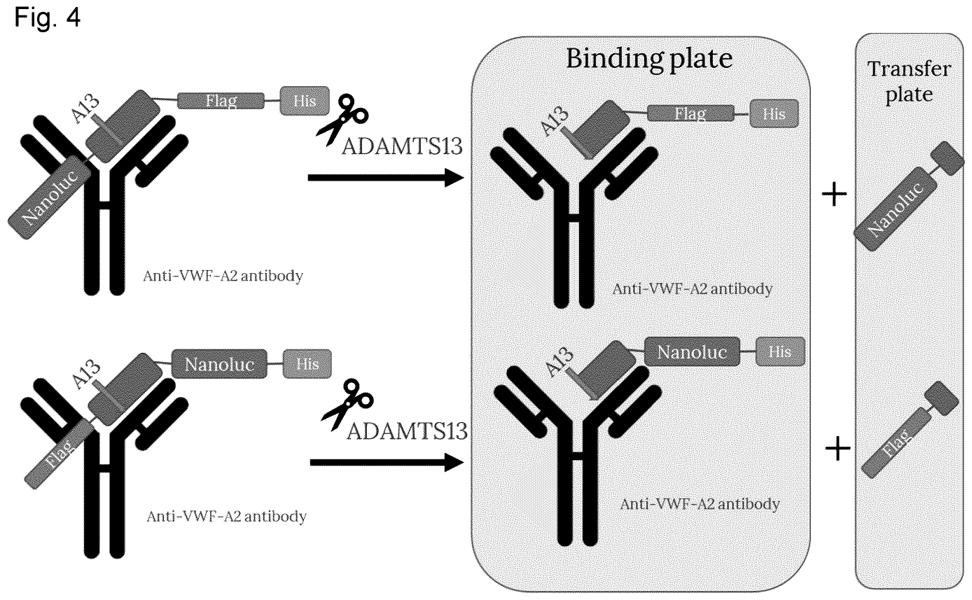

As described in Example 2, polypeptides with N-terminal luciferase domains are advantageous for use with antibodies. A schematic representation of such a strategy is shown in FIG. 4. When the antibody is coated on a binding plate, the polypeptide of the invention is also bound to the plate. After ADAMTS13 cleavage, the luciferase domain can freely enter the solution. Such a supernatant with free luciferase domain can be transferred to another plate and quantified there. For improved sensitivity both the binding plate and the transfer plate can be assayed, for this gives indications of how much polypeptide was cleaved (transfer plate) or was not cleaved (binding plate).

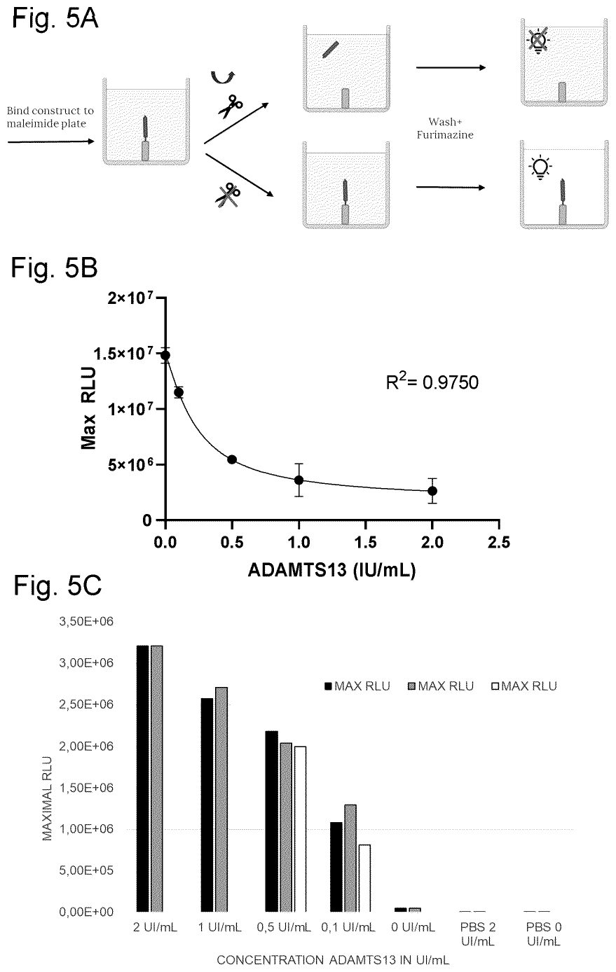

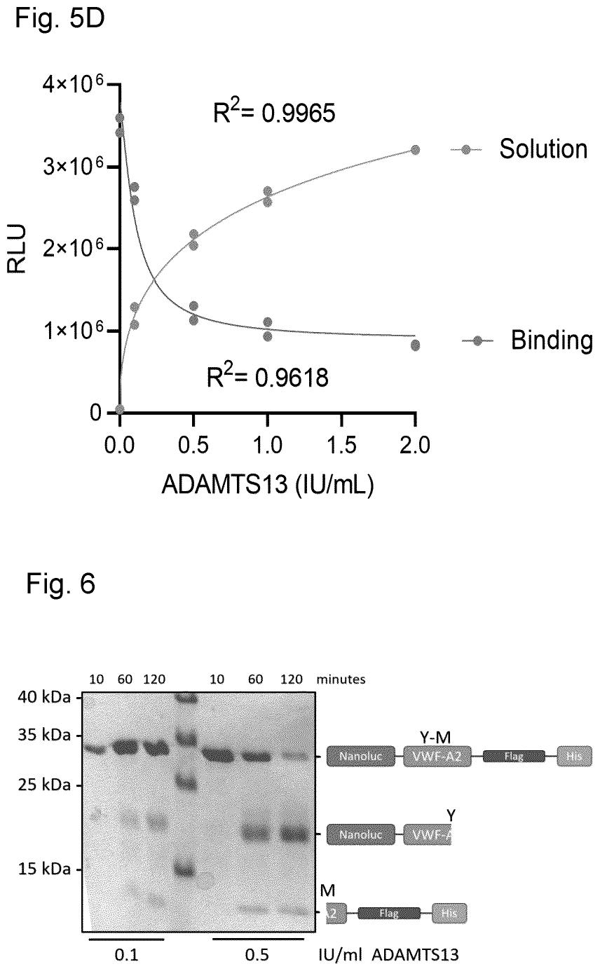

In an alternative strategy that does not require antibodies for immobilisation, a cysteine residue was added to the polypeptide, towards the C-terminus. Examples of such polypeptides are represented by SEQ ID NOs: 17 and 18, which feature (from N to C-terminus) a luciferase domain, a linker, an A2 domain, another linker, a FLAG-tag, the cysteine, and a his-tag. The inventors found that it was essential for this particular strategy to modify the luciferase domain by substituting at least one amino acid, in this case a cysteine residue at position 166 was replaced by a phenylalanine residue. Use is shown in FIG. 5A. The polypeptides are covalently bound to commercially available maleimide plates (8 μg/mL). After coating, unreacted maleimides are blocked using 2 mM D-cysteine for 1 hour, after which a specific binding sites are blocked using 1×PBS with 1% BSA for 1 hour.

For an assay, the coated plates were then incubated with A13 (at concentrations ranging from 2 IU/mL to 0 IU/mL) for 1 hour. Plates were washed, and luminescence was measured with addition of furimazine in 25 mM HEPES with 125 mM NaCl and 0.5% BSA. Results are shown in FIG. 5B, FIG. 5C, and FIG. 5D.

Good results were also obtained with maleimide-coated plastic surfaces, where SEQ ID NO: 17 was coated after which it was contacted with samples containing 1, 0.1 and 0 IU/mL ADAMTS13. NPP and ADAMTS13 deficient plasma, both diluted ten times in 0.5% BSA, 25 mM HEPES, 125 mM NaCl (pH 7.4) buffer, were also tested. Liberated luciferase was contacted with furimazine dissolved in ethanol, diluted in 0.5% BSA, 25 mM HEPES, 125 mM NaCl, 10% sucrose buffer (pH 7.4) to a concentration of 90 μg/mL. There was a clear difference between samples that contain ADAMTS13 and samples without ADAMTS13. The maximum RLU signal output increased with increasing recombinant ADAMTS13 spiked in buffer. Similar results were obtained when normal pooled plasma was tested with normal ADAMTS13 levels, which resulted in a higher signal output compared to an ADAMTS13 deficient plasma sample that does not contain active ADAMTS13 (FIG. 11). These results demonstrate that the signal is low in the absence of ADAMTS13. This suggests that the coupling of the fusion protein to the surface can be successfully used in assays. In the presence of active ADAMTS13, the luciferase part of the fusion protein is liberated and can be used in a luminescent assay. This is shown with an increasing signal output.

### Example 4—Provision of Polypeptides and Further Assay Types

### 4.1 Provision of Polypeptides

DNA fragments encoding the VWF-A2 domains of human von Willebrand Factor (residues 1574-1668), a GlySer spacer domain, the Nanoluc™ (residues 1-170), a FLAG epitope (DYKDDDDK) and a poly-Histidine coding region (Twistgene Inc; www.twistbioscience.com) were amplified by PCR and sub-cloned into the pET28a(+) vector and amplified using E. coli Top10. The target vector was obtained by miniprepping (Qiagen QiAprep Spin Miniprep Kit). Also the Nanoluc sequence was improved for structural stability by reducing conformational entropy3,4, i.e., each glycine residue was substituted to alanine, and demonstrated beneficial (~1.5-fold improved luminescence) changes (G15A, G35A, G51A, G67A, and G71A). The generated plasmids were sequenced to check the appropriate DNA inserts.

The target vectors were transformed into E. coli BL21 (DE3) and grown overnight at 37° C. on LB agar plates containing kanamycin (50 ρg/mL), two colonies were inoculated in LB media (20 mL) containing kanamycin (50 ρg/mL) and grown overnight. LB media (two times 1 L) was inoculated with the cultures and grown at 37° C. (OD400=0.6), isopropyl-1-thio-D-galactopyranoside (IPTG) was added to obtain 1 mM concentration and incubated overnight at 16° C. The bacteria were harvested by centrifugation (2×15 min at 4° C., 5000 rpm), resuspension on ice in 40 mL lysis buffer (25 mM HEPES, 500 mM NaCl, 30 mM imidazole, pH 7.4) followed by sonication on ice (8× (30 sec sonication, 90 sec rest)). Centrifugation (45 min at 4° C., 10000 rpm) obtained the crude fusion protein in the supernatant. A Ni²⁺ NTA column (GE Health Care) was equilibrated with lysis buffer (10 column volumes), charged with the supernatant, flushed with wash buffer 1 (25 mM HEPES, 500 mM NaCl, 60 mM imidazole, pH 7.4) and wash buffer 2 (25 mM HEPES, 500 mM NaCl, 100 mM imidazole, pH 7.4). The fusion protein was eluted with elution buffer (25 mM HEPES, 500 mM NaCl, 250 mM imidazole, pH 7.4).

The purity was monitored by SDS-PAGE on a 4-15% gradient polyacrylamide gel (Bio-Rad) and stained with Coomassie Blue staining (FIG. 6) and Western blot. Fractions containing the pure protein were pooled and subjected to dialysis (25 mM HEPES, 250 mM NaCl). Protein concentrations were determined using the Nanodrop DeNovix DS-11 spectrophotometer and concentrated by a Vivaspin™ column (5 mL, MW 10,000). Degree of cleavage was shown to be dose and time dependent, as expected (FIG. 10).

### 4.2. Development of a Benchtop Assay by Standard ELISA Plate Using the FLAG-Taa

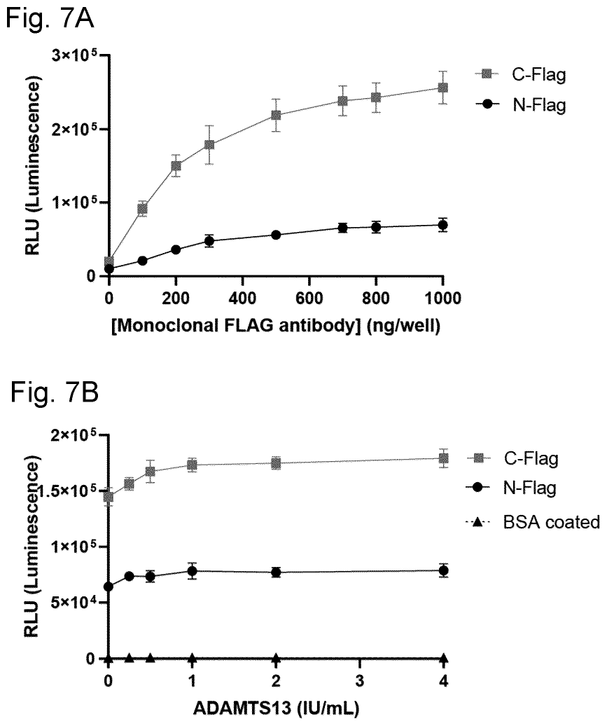

A Corning™ 96 well ELISA plate #3590, transparent, high-binding plate was used to coat a mouse monoclonal anti-FLAG antibody (directed against peptide sequence DYKDDDDK, SEQ ID NO: 24) (M2, F3165-1 mg—SigmaAldrich—lot SLCG2330) in amounts up to 1000 ng/well overnight at room temperature (RT) in PBS. Subsequently, uncoated wells were blocked with 3% BSA blocking solution and washed using 0.1% Tween (Tw-PBS). Subsequently, the fusion product is loaded on the capture antibody in concentrations up to 10 μg/mL. After washing with washing buffer (25 mM Hepes 125 mM NaCl pH 7.4+1% BSA+13 mM CaCl₂)+0.1% Tween) the fusion protein are digested by using A13 concentrations varying from 0-2 IU/mL in incubation buffer (25 mM Hepes 125 mM NaCl pH 7.4+1% BSA+13 mM CaCl₂)). After 1 h digestion at 37° C. with A13, the Nluc domain is released from the fusion protein and 75 μL is transferred to a half area white Greiner Bio-One™ 96 well plate #675075 and added to 25 μL of 170 μM coelenterazine (Carbosynth Ltd, UK). Upon transfer, the plates are immediately transferred to the FLEX station plate reader in luminescence mode (Molecular Devices). Luminescence (in RLU) was measured every 30 s for five minutes. FIG. 7A shows binding of 1000 ng polypeptide to variable concentrations of FLAG antibody, and FIG. 7B shows release of luciferase domain upon A13 cleavage. It was found that for this application, N-terminal A2-domain is preferred because of better signals.

Both fusion proteins show excellent luminescent properties and demonstrate a linear relationship in the fusion protein concentration range from 0.1-100 ng/well in both white luminescent and transparent plates. The data from the transparent plate are useful to establish the amount of fusion protein immobilized by the FLAG antibody and the data from the white luminescent plate are useful to determine the released Nanoluc activity after A13-mediated cleavage of the fusion protein.

FIG. 7A demonstrates that the optimal concentration of monoclonal FLAG antibody is ~400 ng/well and adding 1000 ng/well fusion protein results in a luminescent signal of ~2.5M RLU (C-Flag, SEQ ID NO: 16), demonstrating that the capture antibody has bound ~180 ng/(~18% of the initially offered quantity to the wells), indicating that the binding of the fusion protein to the capture antibody is effective. N-flag (SEQ ID NO: 15) demonstrates less binding to the FLAG antibody of ~0.7M RLU (C-Flag), resembling ~50 ng/(~5% of the initially offered quantity to the wells) and thereby indicating that the N-flag is a less effective construct for setting up an A13 activity test.

FIG. 7B demonstrates minimal cleavage of the fusion protein by A13 in the range from 0-1 IU/mL resulting in a signal increase from ~1.5 towards ~1.7 M RLU for C-Flag, indicating that the current setup may be able suitable setup for further optimization to assess A13 susceptibility of the fusion proteins. Comparison of the 1.7M RLU result from FIG. 7B with luminescence in a white plate showed that ~120 ng VWFA2-Nanoluc (~12% of the initially offered quantity to the wells) was cleaved off (or dissociated from the capture antibody) and transferred to the new white plate.

### 4.3 Development of a Benchtop Assay by Using a His-Tag

A Pierce™ 96 well Nickel coated plate, white #15242, high-binding plate was incubated for 1 h at RT using a C-terminal Flag VWF A2 fusion protein (SEQ ID NO: 16) in amounts up to 400 ng/well in PBS with 3% BSA and 0.1% Tween20. After washing with three volumes of assay buffer (25 mM Hepes, 125 mM NaCl, 15 mM CaCl₂), 0.1% Tween20 and 1% BSA) concentrations of 0-100% recombinant ADAMTS13 is incubated at 37° C. for 1 h. 75 μL of the cleaved off Nluc is transferred to a half area white Greiner Bio-One™ 96 well plate #675075 labelled ‘solution plate’ and 25 μL coelenterazine is added to final concentration of 40 μM. The remaining fusion proteins are washed three times and 75 μL of PBS with 25 μL coelenterazine is added. This plate is referred to as ‘binding plate’.

Upon transfer of the coelanterazine, the plates are immediately transferred to the SpectraMax M3 plate reader in luminescence mode (Molecular Devices). Luminescence (in RLU) was measured every 30 s for five minutes.

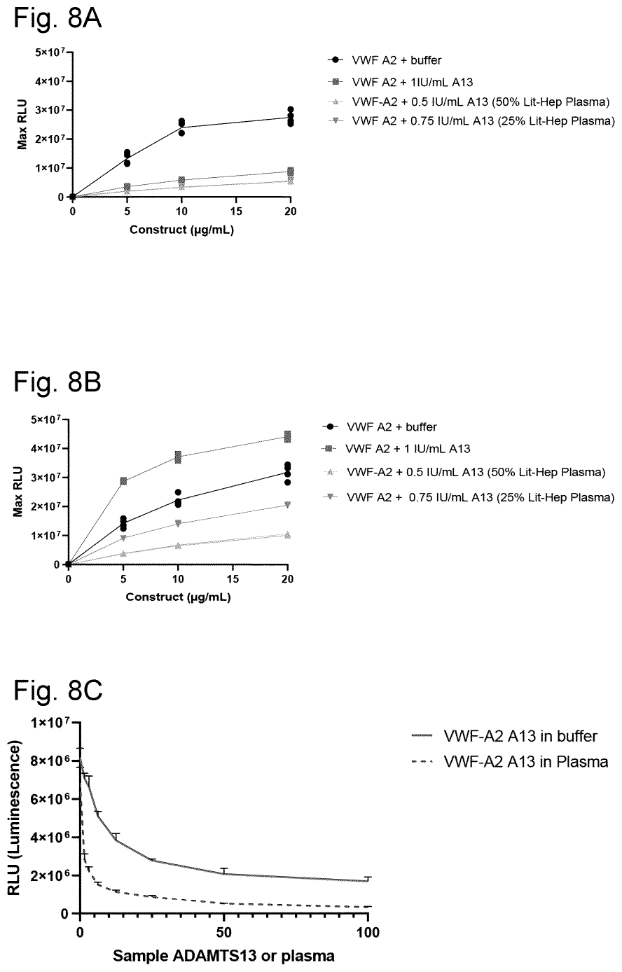

The C-Flag fusion proteins show excellent binding to the Ni²⁺-NTA plate with a linear relationship up to 10 μg/mL and saturation of the Ni2+-NTA plate in concentrations above (FIG. 8A). The maximal luminescence intensity in buffer yields a signal of 2.5×10⁷ RLU. Upon incubation with A13 either in buffer or in various concentration of Lithium-heparin plasma for 1 h at RT the Nluc is cleaved off by either the recombinant or plasma derived A13 and therefore the signal reduces. From the data with 0.5 and 0.75 IU/mL A13 in lithium heparin plasma it appears that the proteins in plasma also reduce the binding to the Ni²⁺-NTA plate (i.e. a matrix effect).

FIG. 8B demonstrates Nanoluc luminescence after transfer of the cleaved fusion proteins to the solution plate. These data demonstrate a significant transfer of VWF-A2 fusion protein after incubation in buffer, but even higher for the incubation with 1 IU/mL A13 which may indicate non specific release from the Ni-NTA plate. On the other hand, when the fusion protein is incubated with 0.50 and 0.75 IU/mL A13 in lithium heparin plasma it appears that the reduced binding on the binding plate in FIG. 8A leads to diminished signal in the solution plate possibly caused by the inhibitory capacity of the plasma matrix or the negatively charged heparins in particular.

In FIG. 8C we used both the VWF-A2 fusion protein and a VWF-D3 fusion protein that is not susceptible for A13 cleavage and therefore serves as a negative control in this A13 assay setup. When VWF-A2 fusion protein is bound to the plate in buffer high signals are found of ~8×10⁶ RLU and upon cleavage by A13 the signals rapidly decrease and stabilise at ~2×10⁶ RLU. When adding similar amounts of A13 from lithium-heparin plasma the signal drop even faster and stabilize at around 0.4×10⁶ RLU again indicating that, next to the A13 effect, the plasma matrix or the negatively charged heparins may play a role. The VWF-D3 fusion protein that serves as a negative control appear to remain largely unaffected by the A13 activity, since the signal intensity reduces from ~5 to ~4×10⁶ RLU. In plasma however the signal was reduced from ~4 to ~2×10⁶ RLU which indeed may be an indication for a plasma matrix effect.

These results indicate that the A13 assay is highly sensitive in the lower concentrations ranging from 0-30% A13 activity both in a buffer and lithium-heparin blood plasma environment. Together with the extremely high signal intensities of 8×10⁶ RLU this makes the current setup especially suitable for further development into a microfluidic cartridge format.

1. Polypeptide comprising a first sequence and a second sequence, wherein the first sequence encodes a polypeptide that has luciferase activity, and wherein the second sequence encodes a von Willebrand factor (VWF) domain that comprises a tyrosine-methionine recognition site for ADAMTS13.

2. The polypeptide according to claim 1, wherein the first sequence encodes a polypeptide that has the activity of Enhanced Beetle Luciferase (ELuc), Click Beetle Green Luciferase (CBG), R. ohbai Luciferase (RoLuc), Firefly Luciferase (FLuc), Red Firefly Luciferase (RedF), P. hirtus Red Luciferase (RedLuc), Nano Luciferase (NLuc), Renilla Luciferase (Renilla), Metridia Luciferase (MetLuc), Lucia Luciferase (Lucia) Gaussia Luciferase (GLuc), or Green Renilla Luciferase (GrRenilla), preferably it has the activity of NLuc.

3. The polypeptide according to claim 1, wherein the first sequence has at least 70% sequence identity with any one of SEQ ID NOs: 1-12.

4. The polypeptide according to claim 1, wherein the second sequence has at least 70% sequence identity with any one of SEQ ID NOs: 13-14.

5. The polypeptide according claim 1, wherein the second sequence encodes the A2 domain of VWF, wherein the tyrosine-methionine recognition site for ADAMTS13 comprises a tyrosine at position 105, 106, 107, 108, 109, 110, or 111, wherein that tyrosine is directly followed by a methionine, optionally wherein the tyrosine-methionine recognition site is directly flanked by two valine residues,
or wherein the second sequence encodes part of the A2 domain of VWF, wherein the tyrosine-methionine recognition site for ADAMTS13 comprises a tyrosine at position 29, 30, 31, 32, 33, 34, or 35, wherein that tyrosine is directly followed by a methionine, optionally wherein the tyrosine-methionine recognition site is directly flanked by two valine residues.

6. The polypeptide according claim 1, wherein the first sequence is N-terminal to the second sequence, or wherein the second sequence is N-terminal to the first sequence, preferably wherein the second sequence is N-terminal to the first sequence.

7. The polypeptide according claim 1, wherein the polypeptide comprises a third sequence that encodes a first recognition tag,
and optionally comprises a fourth sequence that encodes a second recognition tag, wherein the second recognition tag is distinct from the first recognition tag.

8. The polypeptide according to claim 7, wherein the recognition tag is an epitope tag, an affinity-tag, a cysteine residue, or an aptamer-tag.

9. The polypeptide according to claim 1, wherein the first sequence and the second sequence are separated by a linker sequence, wherein the linker sequence comprises 1-40 amino acids.

10. The polypeptide according to claim 1, wherein the polypeptide comprises 200-500 amino acids,
optionally wherein the first sequence has a length of 150-450 amino acids,
optionally wherein the second sequence has a length of 50-200 amino acids,
optionally wherein a third sequence that encodes a first recognition tag is present and has a length of 4-25 amino acids,
optionally wherein the fourth sequence that encodes a second recognition tag is present and has a length of 4-25 amino acids,
optionally wherein one or more linkers are present and each linker sequence has a length of 1-30 amino acids.

11. The polypeptide according to claim 1, wherein the polypeptide comprises
a third sequence that encodes a first recognition tag, and
a fourth sequence that encodes a second recognition tag, wherein the second recognition tag is distinct from the first recognition tag, and preferably
wherein the order of the sequences within the polypeptide, from N-terminus to C-terminus, is either
i) the third sequence, the second sequence, the first sequence, and the fourth sequence; or
ii) the first sequence, the second sequence, the third sequence, and the fourth sequence.

12. The polypeptide according to claim 1, wherein the polypeptide has at least 95% sequence identity to SEQ ID NOs: 15-18, preferably to SEQ ID NO: 15, 17, or 18, preferably at least 98% sequence identity.

13. Nucleic acid construct comprising a sequence that encodes a polypeptide as defined in claim 1.

14. (canceled)

15. Method for quantifying ADAMTS13 in a sample, the method comprising the steps of:
a) contacting the sample with a composition comprising a polypeptide as defined in claim 1 to release a polypeptide fragment with luciferase activity; wherein the polypeptide is preferably freely dissolved;
b) separating the polypeptide fragment with luciferase activity from the remainder of the polypeptide as defined in claim 1;
c) contacting the fragment with luciferase activity with a suitable substrate; and
d) determining the relative light intensity generated by the fragment with luciferase activity.

16. The polypeptide according to claim 1, wherein the second sequence has at least 90% sequence identity to SEQ ID NO: 52.

17. The polypeptide according to claim 1, wherein the polypeptide has at least 90% sequence identity to SEQ ID NO: 15.

18. The polypeptide according to claim 1, wherein the polypeptide has at least 90% sequence identity to SEQ ID NO: 17.

19. The polypeptide according to claim 1, wherein the polypeptide has at least 90% sequence identity to SEQ ID NO: 54.

20. The polypeptide according to claim 1, wherein the polypeptide has at least 90% sequence identity to SEQ ID NO: 56.
